(19) United States
(12) Patent Application Publication
(10) Pub. No.: US 2026/0257054 A1
(43) Pub. Date: Sep. 3, 2026

(54) CENTRIFUGAL LVAD WITH WIRELESS POWER TRANSFER AND ANTITHROMBOTIC SLIC COATING

(71) Applicants: GEORGIA TECH RESEARCH CORPORATION, Atlanta, GA (US); NORTH CAROLINA STATE UNIVERSITY, Raleigh, NC (US); WILLIAM MARSH RICE UNIVERSITY, Houston, TX (US); TEXAS HEART INSTITUTE, Houston, TX (US); BAYLOR COLLEGE OF MEDICINE, Houston, TX (US)

(72) Inventors: Lakshmi Prasad DASI, Atlanta, GA (US); Huang CHEN, Atlanta, GA (US); O.H FRAZIER, Houston, TX (US); Shweta KARNIK, Atlanta, GA (US); Arun Kumar KOTA, Raleigh, NC (US); Yaxin WANG, Houston, TX (US); Joseph CAVALLARO, Houston, TX (US); Simon KIANG, Houston, TX (US); Antonio Mendoza GONZALES, Houston, TX (US)

(21) Appl. No.: 19/489,482

(22) PCT Filed: Jun. 3, 2024
(86) PCT No.: PCT/US2024/032278

§ 371 Date: Dec. 2, 2025

Related U.S. Application Data
(60) Provisional application No. 63/505,873, filed on Jun. 2, 2023.

(51) Int. Cl. A61M 60/17; A61M 39/00; A61M 39/10; A61M 60/232; A61M 60/419; A61M 60/816; A61M 60/82

(52) U.S. Cl. CPC A61M 60/17; A61M 39/10; A61M 60/232; A61M 60/419; A61M 60/816; A61M 60/82; A61M 2039/0009; A61M 2039/1027; A61M 2205/0238; A61M 2205/04; A61M 2205/103; A61M 2205/3303; A61M 2205/3327; A61M 2205/3523; A61M 2205/50; A61M 2205/8206; A61M 2205/8243; A61M 2207/00; A61M 2210/125; A61M 2230/005; A61M 2230/06

(57) ABSTRACT

An exemplary left ventricular assist device (LVAD) and method that employs design and material designs to reduce the thrombosis risk of LVAD implant in a patient. One feature of the design includes a stented inlet member for the device that can reduce or fully eliminate a flow stasis that can trigger blood protein adsorption that can lead to chain reactions that then result in thrombosis. Other features of the design include (i) a flexible rotor and/or pump housing that can reduce blood damage, (ii) hydrophilic slippery coatings at high sheer components, such as the rotor to further reduce protein adsorption and thus thrombosis risk, (iii) magnetic-based drive and bearing components that can improve the hemocompatibility of the blood pump, and (iv) advanced controls and charging.

### RELATED APPLICATION

This PCT application claims priority to, and the benefit of U.S. Provisional Patent Application No. 63/505,873, filed Jun. 2, 2023, entitled, “CENTRIFUGAL LVAD WITH WIRELESS POWER TRANSFER AND ANTITHROMBOTIC SLIC COATING,” which is incorporated by reference herein in its entirety.

### BACKGROUND

When cardiovascular disease and heart failure (HF) reach the end stage, no known medication is effective in treating them. Left ventricular assist device (LVAD) implants are often used to help a failing heart to sustain blood circulation to the body when donor hearts are not available. Common complications for LVAD devices are known to include infection, thrombosis, stroke, and bleeding after the transplant, any of which can greatly reduce the quality of life for the LVAD patient.

Blood damage caused by the mechanical force from LVAD devices has been known to cause adverse effects, including bleeding and thrombosis, requiring rehospitalization and emergency surgery. The blood damage is often associated with the supraphysiological shear stress in an LVAD device from mechanical force destroying blood cells, leading to hemolysis, activating platelets, and causing thrombosis. There is currently one LVAD device commercially available.

There is a benefit to improving left ventricular assist devices.

### SUMMARY

An exemplary left ventricular assist device (LVAD) and method are disclosed that employs design and material designs to reduce the thrombosis risk of LVAD implant in a patient. One feature of the design includes a stented inlet member for the device that can reduce or fully eliminate a flow stasis that can trigger blood protein adsorption that can lead to chain reactions that then result in thrombosis. Thrombosis is the formation of a blood clot in a blood vessel or the heart that can block or direct blood flow appropriately. Another feature of the design includes a flexible rotor and/or pump housing that can reduce blood damage. The design was observed to improve the pump efficiency hydrodynamically. Another feature of the design employs hydrophilic slippery coatings at high sheer components, such as the rotor, to further reduce protein adsorption and thus thrombosis risk. Another feature of the design employs magnetic-based drive and bearing components that can improve the hemocompatibility of the blood pump. Each and the discussed combined features can reduce the associated risk of LVAD to make them more available to more critical patients and change the therapy options for cardiovascular disease and heart failure (HF).

The exemplary LVAD can further include system improvements and improvements in controls, leveraging advanced controls and electronics and advanced charging and energy management controls to improve device usability, reduce the likelihood of premature failure, and enhance device operation. In some embodiments, a wireless power transfer and communication submodule can be implemented to eliminate the percutaneous drivelines that have been associated with severe infections. The various mechanical, electrical, material, and control system features can be employed, in whole or in part, in a new LVAD, or it can be an existing commercial LVAD design. An LVAD employing some or all of the noted features can (i) support patients for a longer period of time with fewer anticoagulant treatments and (ii) operate with a lower risk for emergency surgeries to treat complications. An LVAD optimized with all of the noted features, among others, can potentially serve a viable treatment non-inferior to existing cardiac transplants.

An exemplary AI-based algorithm and analysis is disclosed, employing state-of-the-art deep learning algorithms to analyze the relationship between different geometry parameters and blood damage in the design space. AI-based algorithms can be used to evaluate suitable patients for the LVAD device or therapy.

In addition to LVAD, the various mechanical, electrical, material, and control system features may be employed for other medical implants as well as devices. Examples of such devices include but are not limited to extracorporeal membrane oxygenations (ECMO) and cardiopulmonary bypass (CPB) machines. Cardiopulmonary Bypass is a machine that temporarily takes over the function of the heart and lungs during cardiac surgery by maintaining the circulation of blood and oxygen throughout the body. Extracorporeal membrane oxygenation is a form of extracorporeal life support, providing prolonged cardiac and respiratory support to persons whose heart and lungs are unable to provide an adequate amount of oxygen, gas exchange, or blood supply to sustain life.

The anti-thrombotic SLIC coatings can be employed for blood-contacting devices such as catheters, guidewires, dialyzers, oxygenators, heart-supporting systems, cardiac pacemakers, vascular grafts, stents, heart valves, etc., to reduce such thrombotic risk. In some embodiments, less invasive treatments may be employed. The exemplary SLIC coatings may reduce thrombosis in blood-contacting devices and materials as well as fouling-free coatings devices, e.g., for contact lenses, chemical and biomedical sensors, food and beverage equipment, marine equipment, etc.

In an aspect, an implantable left ventricular assist device (LVAD) is disclosed comprising a pump housing forming a volume therein, the pump housing having an inlet and an outlet; a rotor disposed within the pump housing to rotate in the volume to pump blood; a motor coupled to the rotor to drive rotation of the rotor; and an inlet member coupled to the inlet of the housing, the inlet member having (i) an external structure configured to conform and contact an outer surface wall at a base of a left ventricle and (ii) an internal expandable body configured to move between a stowed configuration and a deployed configuration to be placed and extending into the left ventricle, wherein the internal expandable body is defined by (i) a first section having a first circumference sized to couple to the inlet of the pump and (ii) a second section having a second circumference sized, wherein in the deployed configuration, to substantially contact an inner surface wall of the left ventricle to eliminate flow stasis in the left ventricle.

In some embodiments, the rotor comprises a rotor body and a plurality of curved flexible blades extending therefrom, wherein the plurality of curved flexible blades are flexibly shaped and formed of a flexible material (e.g., polyurethane rubber, etc.) to reduce shear stress on components of the blood (e.g., while also increasing pump efficiency).

In some embodiments, the rotor comprises an antithrombotic coating (e.g., configured to reduce protein adsorption and thrombosis risk) comprising a hydrophilic and slippery polymer.

In some embodiments, the device includes a drive system configured to drive the rotor to rotate in the volume of the pump housing, and an implanted control unit that electrically couples to the drive system to control the drive system and rotation of the rotor, wherein the implanted control unit includes: one or more energy storage devices; a charging circuit; and a wireless power transfer circuit comprises an antenna coil and electronics to control inductive charging operation with an external RF source (e.g., transfer circuit allows for no percutaneous drivelines that can cause infections).

In some embodiments, the motor comprises a maglev drive subassembly comprising: one or more permanent magnets (e.g., radial passive magnets, e.g., located at an inlet cannula portion of the rotor body) located in the rotor body; a stator disposed at a position in the pump housing proximal to the one or more permanent magnets (e.g., wherein the stator includes a permanent magnet, wherein the permanent magnets of the stator and one or more permanent magnets in the rotor body repulsive force between the rotor and stator permanent magnets to stabilize the radial direction and inclination passively); and one or more magnetic bearings disposed in one or more positions in the pump housing proximal to the plurality of curved flexible blades of the rotor (passive magnetic levitated bearing located at the bottom of the VAD housing).

In some embodiments, the internal, expandable body of the inlet member comprises the flexible mesh surrounded by or embedded in a fabric, wherein the flexible mesh is configured as self-expanding or balloon-expanding.

In some embodiments, the internal expandable body of the inlet member is antithrombotic and anti-restenotic drug-eluting.

In some embodiments, after deployment, the internal, expandable body is defined by (i) a first section having a first circumference sized to couple to the inlet of the pump and (ii) a second section having a second circumference sized to continuously contact and conform to the inner surface wall of the left ventricle, to conform to a shape of the left ventricle.

In some embodiments, the internal, expandable body (and external surface) is configured to deform with ventricular motions over a pre-defined set of deformation cycles (e.g., to resist the failure caused by fatigue).

In some embodiments, the fabric comprises a resorbable material to promote endothelialization (e.g., absolvable over time to only leave neo-tissues).

In some embodiments, the internal, expandable body comprises a resorbable material to promote endothelialization (e.g., absolvable over time to only leave neo-tissues).

In some embodiments, the fabric includes polyester (e.g., PET such as Dacron), polytetrafluoroethylene (e.g., expanded polytetrafluoroethylene (ePTFE)), biological tissue (e.g., pericardium tissue), or a combination thereof.

In some embodiments, the internal, expandable body, including flexible mesh and fabric, is patient-specifically designed to be sized and shaped to a configuration to match a scan of the left ventricle of a patient (e.g., the scan being computationally modeled and a portion of the internal, expandable body, e.g., flexible mesh, being 3D printed to accommodate the complex anatomical variants of the ventricle).

In some embodiments, the pump housing and/or the inlet member comprises a quick disconnect connector to releasably and attachably connect the pump housing to the inlet member (e.g., wherein the device can be installed and deployed with the pump housing to the inlet member disconnected, and wherein the inlet member can be capped when desired).

In some embodiments, the pump housing has (i) an external structure and (ii) an internal deformable structure are disposed within a portion of a surface defining the volume of the pump housing, the internal deformable member comprising a flexible material.

In some embodiments, the flexible material of the internal deformable member is the same as the flexible material of the rotor.

In some embodiments, the flexible material of the internal deformable member is different from the flexible material of the rotor.

In some embodiments, the portion of the surface defining the volume of the pump housing having the flexible material corresponds to regions of contact with the plurality of curved flexible blades during rotation of the plurality of curved flexible blades.

In some embodiments, the portion of the surface defining the volume of the pump housing having the flexible material corresponds to regions of contact with the plurality of curved flexible blades during (i) rotation of the plurality of curved flexible blades and (ii) off-axis movement of the plurality of curved flexible blades mounted configured to move off-axis via a magnetic bearing and electromagnetic driving subsystem.

In some embodiments, the external structure is made of flexible material.

In some embodiments, the internal surfaces of the pump housing, including the internal deformable structure comprise an antithrombotic coating (e.g., configured to reduce protein adsorption and thrombosis risk) comprising a hydrophilic and slippery polymer.

In some embodiments, the antithrombotic coating of the interface surfaces of the pump housing and/or the antithrombotic coating of the rotor is optimized for a maximum antithrombotic response via systematic tailoring of hydroxylation parameters, molecular architecture, and synthesis reaction coordinates.

In some embodiments, the antithrombotic coating of the interface surfaces of the pump housing and/or the antithrombotic coating of the rotor is formed via liquid-phase silanization of the flexible material of the interface surfaces of the pump housing and/or the antithrombotic coating of the rotor to form a silane (e.g., for achieving a high degree of substrate hydroxylation by modifying the flexible material surfaces using oxygen plasma).

In some embodiments, the silane is tuned (i) to obtain high grafting densities to the flexible material of the interface surfaces of the pump housing and/or the antithrombotic coating of the rotor and (ii) to ensure an antithrombotic nature.

In some embodiments, the coating is adjusted by synthesis reaction.

In some embodiments, the device further includes an implantable subcutaneous coil, wherein the wireless power transfer circuit of the implanted control unit is operatively coupled via a drive line conductor to the implantable subcutaneous coil, the implantable subcutaneous coil being configured to operate as a pair of coupled coils with an external coil configured to be affixed (i) to a skin region of the patient and (ii) in proximity to the implantable subcutaneous coil.

In some embodiments, the one or more energy storage devices include (i) one or more rechargeable batteries and (ii) at least one of a supercapacitor and a hybrid supercapacitors, the one or more rechargeable batteries and the at least one of the supercapacitor and the hybrid supercapacitors having a combined energy storage of at least 20 minutes (e.g., 30 minutes, 40 minutes, 50 minutes, 1 hour, 2 hour).

In some embodiments, the implanted control unit comprises (i) a processor and (ii) a memory having instructions stored thereon, wherein execution of the instructions by the processor causes the processor to execute a dynamic charging algorithm (e.g., based on internal and external temperature sensors that pulse wireless energy transfer to control heating and charge level).

In some embodiments, the implanted control unit comprises a wireless communication interface configured to operatively connect with a remote controller (e.g., edge device, cloud infrastructure, or a combination thereof).

In some embodiments, the remote controller has (i) a processor and (ii) a memory having instructions stored thereon, wherein execution of the instructions by the processor causes the processor to: execute a dynamic charging algorithm comprising a trained machine learning model or a model derived therefrom (e.g., configured to monitor current patient activity state to determine LVAD demand and immediate flow settings); and transmit an output of the dynamic charging algorithm to the implanted control unit to adjust control operation thereat.

In some embodiments, the implanted control unit is configured to execute a dynamic charging algorithm.

In some embodiments, the dynamic charging algorithm (e.g., local in the LVAD and/or external) includes a dynamic motor control loop that can reduce the LVAD speed during time of less patient activity, the dynamic motor control loop has an output to drive operation of the motor.

In some embodiments, the device includes a sensor configured to acquire an electrical signal (e.g., ECG signal, waveform, or the like) of the heart, wherein the dynamic motor control loop comprises one or more inputs, including a first input to receive the acquired electrical signal.

In some embodiments, the pump housing comprises a position sensor (e.g., eddy current sensor, e.g., configured to measure the gap between the rotor, e.g., impeller tip, and the pump housing) for the rotor, and wherein the one or more inputs of the dynamic motor control loop, includes a second input to receive an acquired electrical signal from the position sensor.

In some embodiments, the trained machine learning algorithm is used to evaluate long-term LVAD performance data (e.g., to tune settings).

In some embodiments, the device further includes a drive system with magnetic levitation that includes a magnetically levitated ventricular assist device (VAD), comprising: an impeller attached to rotor permanent magnets; a stator embedded within the VAD housing adjacent to an inlet cannula; an active magnetic levitated bearing located at the bottom of the VAD housing with an eddy current sensor configured to monitor the gap between the impeller tip and the maglev.

In some embodiments, the implantable left ventricular assist device was selected to be implanted in a patient identified via a trained ML algorithm employed to evaluate candidate assessment.

In some embodiments, the trained ML algorithm is configured to estimate a likelihood of presence of a candidate criteria for an LVAD implant (e.g., Left Bundle Branch Block (LBBB), Premature Ventricular Contraction (PVC), Left Ventricular Hypertrophy (LVH), Anterior Myocardial Infarction (AMI) and Congestive Heart Failure (CHF), QRS duration, Inferior Myocardial Infarction (IMI) and Atrioventricular Block (AVB); Right Ventricular Hypertrophy (RVH), Right Bundle Branch Block (RBBB), Right Atrial Enlargement/Overload (RAE), and a combination thereof).

In some embodiments, the stator forms a brushless DC (BLDC) motor with the rotor, wherein optimization of diameters of stator and rotor is performed via numerical simulation (e.g., to prevent flux saturation at operational speed) and the length of the stator is optimized to generate motor torque to sufficiently meet hydraulic torque requirement.

In some embodiments, the system further includes a drive system with magnetic levitation that includes a magnetically levitated ventricular assist device (VAD), comprising: an impeller attached to rotor permanent magnets; a stator embedded within the VAD housing adjacent to an inlet cannula; an active magnetic levitated bearing located at the bottom of the VAD housing with an eddy current sensor configured to monitor the gap between the impeller tip and the maglev.

In some embodiments, the magnetically levitated ventricular assist device includes a Magnetic Bearing System (MBS) for controlling rotation and translations of the rotor and impeller, wherein radial passive magnets are fixed to the rotatory impeller, and permanent magnets are attached near the inlet cannula, and where the magnets are configured such that the repulsive force between the rotor and stator permanent magnets stabilizes the radial direction and inclination passively.

### BRIEF DESCRIPTION OF THE DRAWINGS

FIG. 1 shows an example left ventricular assist device configured with a stent-inspired inlet member to reduce or fully eliminate a flow stasis, in accordance with an illustrative embodiment.

FIGS. 2A-2D show examples of LVADs with a stented or stent-like inlet member in accordance with an illustrative embodiment.

FIGS. 3A-3G shows flexible rotor and/or pump housing and design parameters for the exemplary LVAD of FIG. 1 in accordance with an illustrative embodiment.

FIGS. 4A-4D show a slippery hydrophilic (SLIC) surface that can spread aqueous droplets in the exemplary LVAD of FIG. 1 in accordance with an illustrative embodiment.

FIG. 5 show an example wireless energy transfer and communication system that can be implemented in the exemplary Centrifugal LVAD of FIG. 1 in accordance with an illustrative embodiment.

FIG. 6 shows an example LVAD configured with a drive system (also referred to as a “maglev system”) with magnetic levitation in accordance with an illustrative embodiment.

FIGS. 7A and 7B show various features of a study related to heartbeat-synchronized speed modulation.

FIGS. 8A-8E show various features of a study related to the evaluation of hydraulic performance of the flexible motor and impeller for an LVAD.

FIGS. 9A-9B show various features of a study related to the evaluation of a slippery hydrophilic (SLIC) surface for an LVAD.

FIGS. 10A-10D show various features of a study related to the evaluation of wireless energy transfer and communication system for an LVAD.

FIG. 11 shows various features of a study related to the evaluation of heartbeat synchronized speed modulation controls for an LVAD.

FIG. 12A-12D show various features of a study related to the evaluation of magnetic drive and bearing system (MagLev) for an LVAD.

FIGS. 13A-13C show various features of a study related to the evaluation of trained AI model to evaluate candidate recipients for an LVAD.

### DETAILED DESCRIPTION

Some references, which may include various patents, patent applications, and publications, are cited in a reference list and discussed in the disclosure provided herein. The citation and/or discussion of such references is provided merely to clarify the description of the disclosed technology and is not an admission that any such reference is “prior art” to any aspects of the disclosed technology described herein. In terms of notation, “[n]” corresponds to the nth reference in the list. For example, [1] refers to the first reference in the list. All references cited and discussed in this specification are incorporated herein by reference in their entirety and to the same extent as if each reference was individually incorporated by reference.

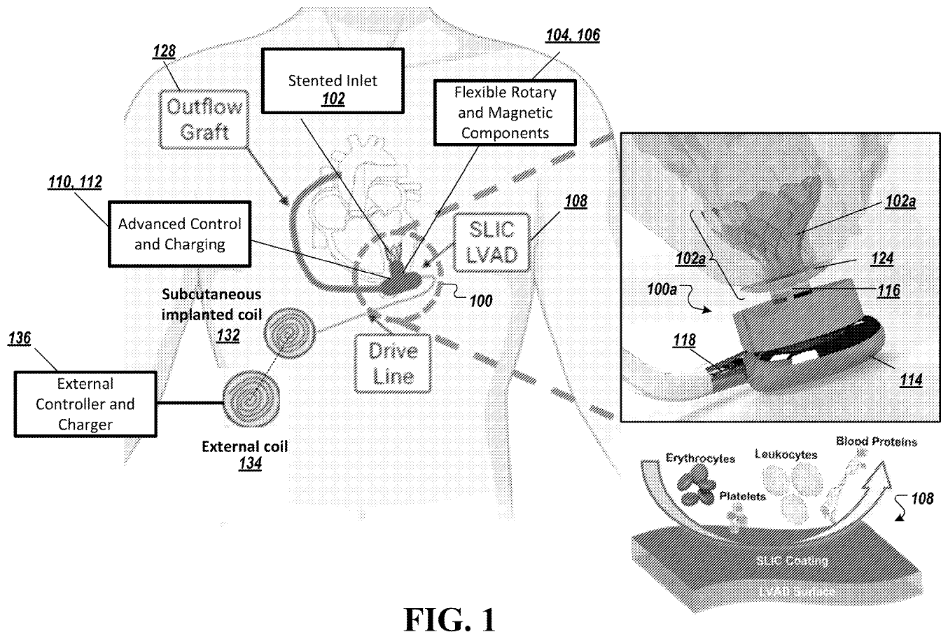

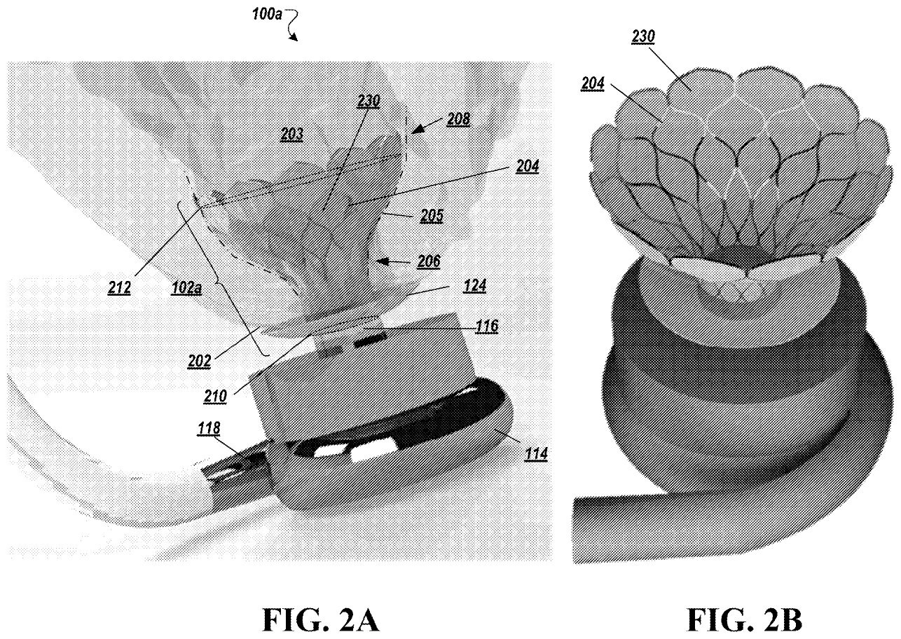

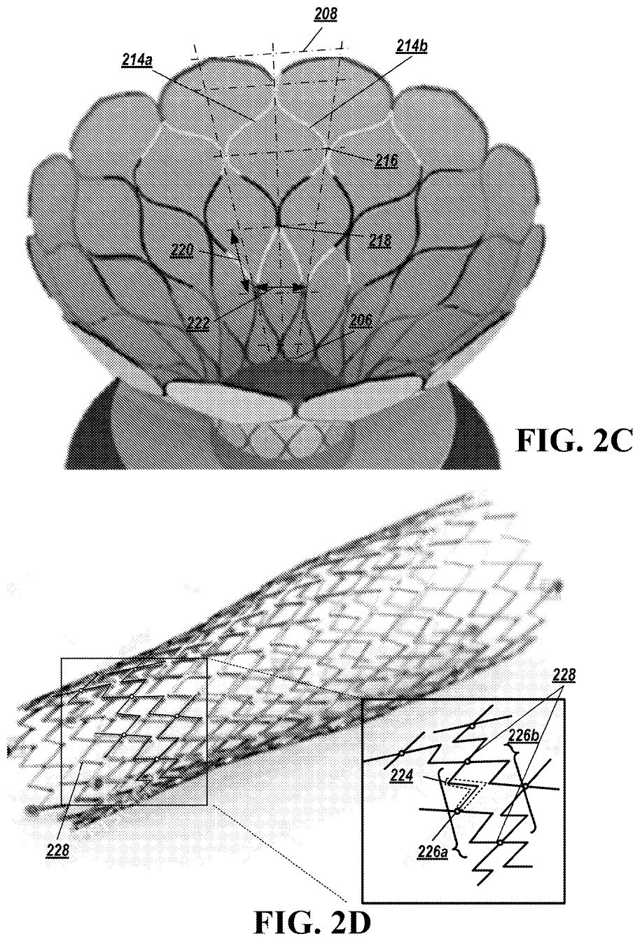

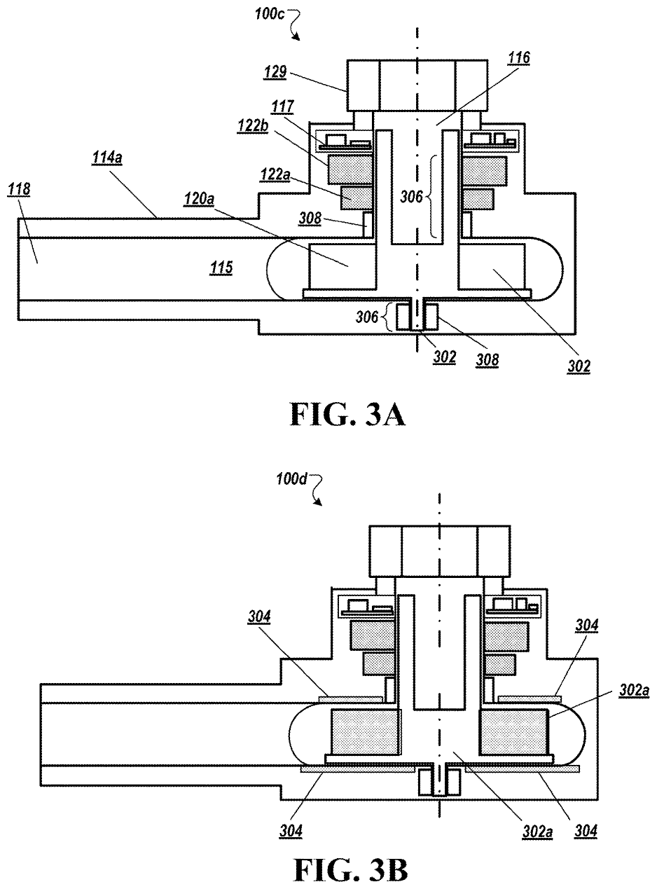

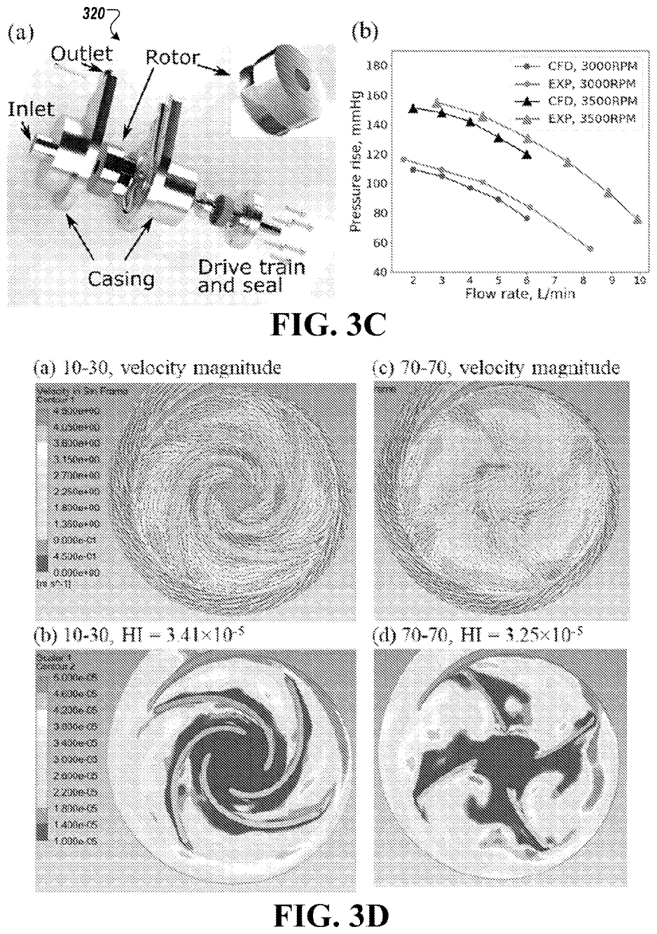

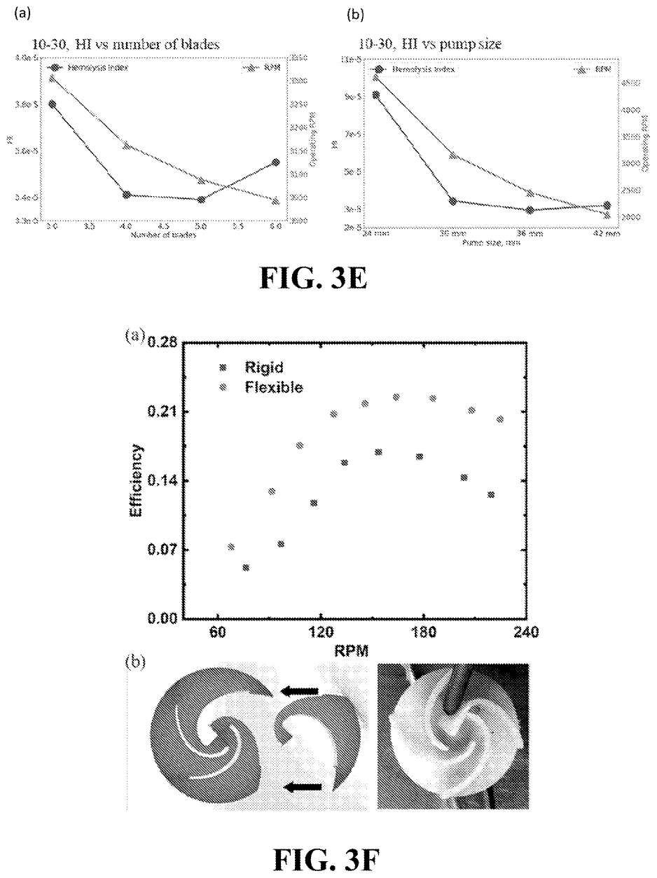

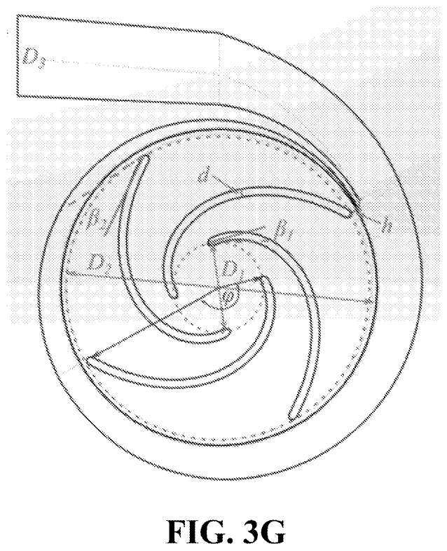

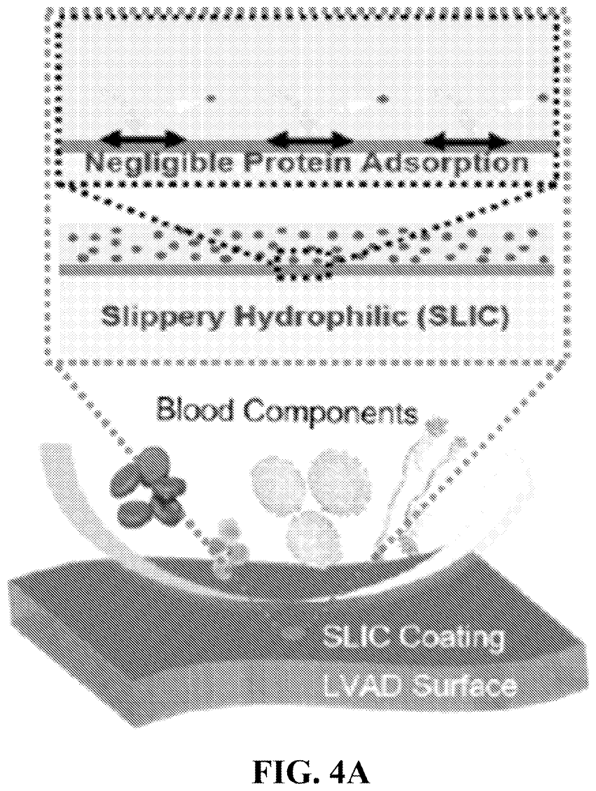

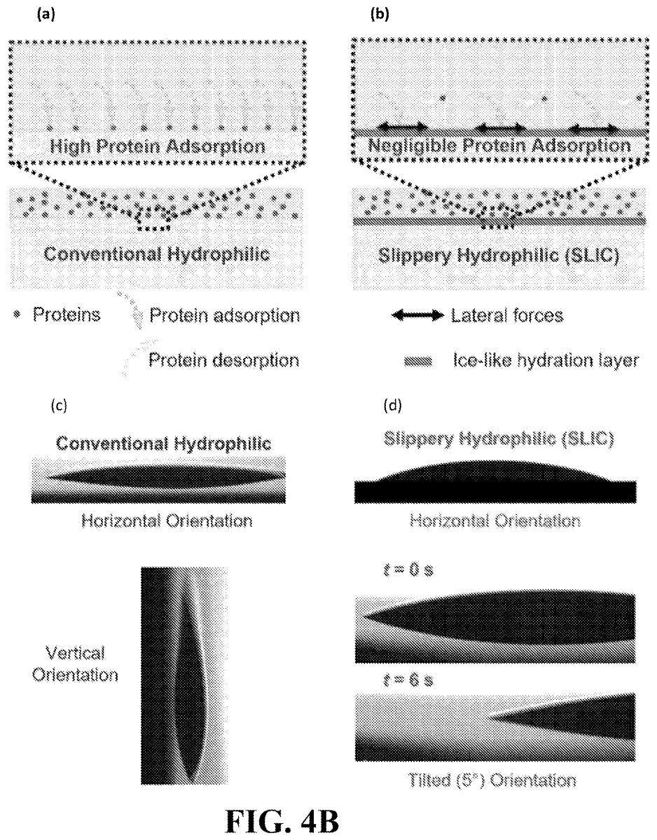

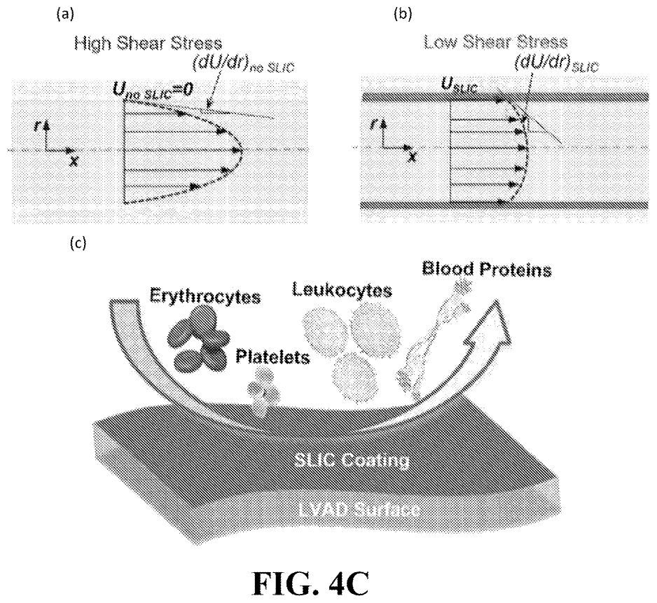

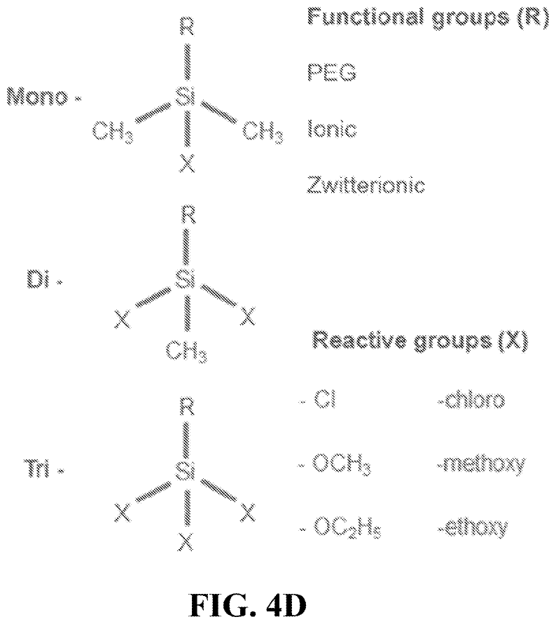

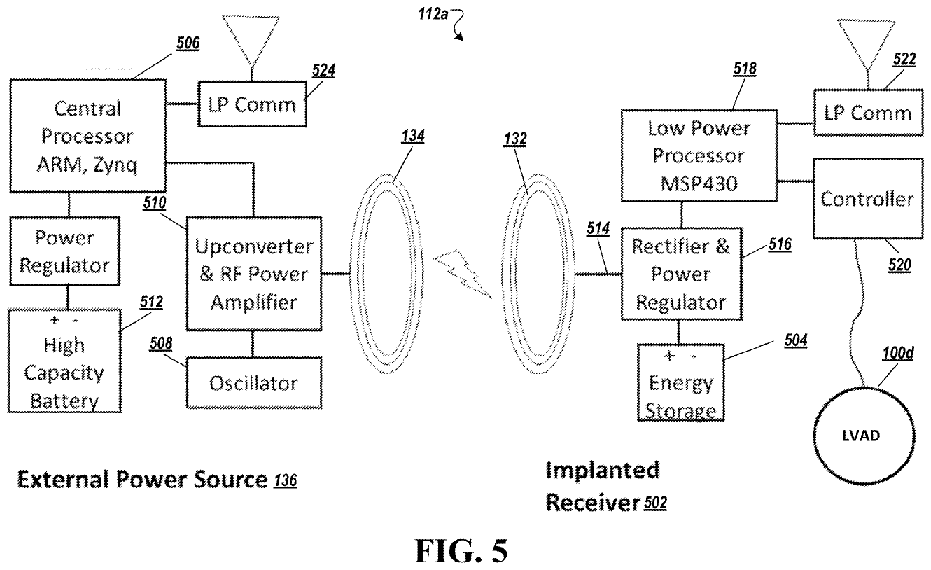

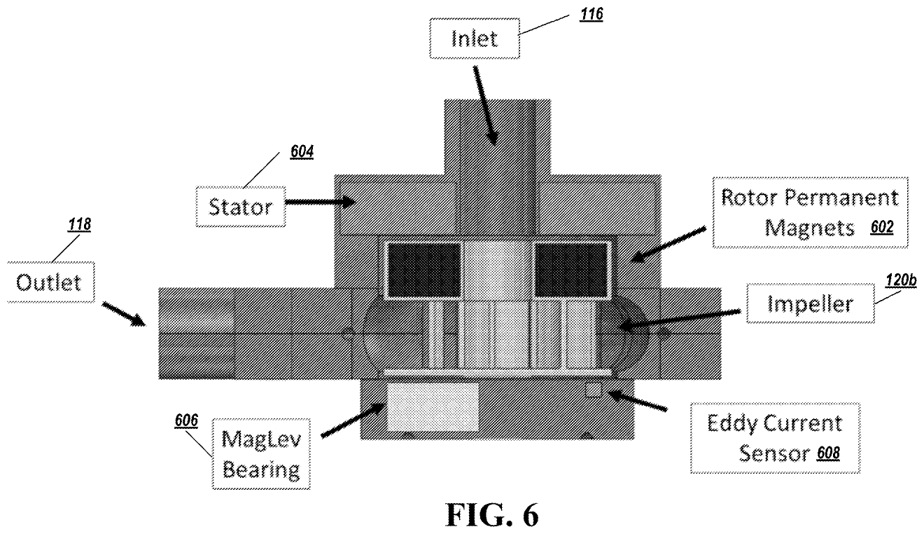

FIGS. 1, 2A, 2B each show an example left ventricular assist device 100 (shown as 100a, 100b) configured with a stent-inspired inlet member 102 (shown as 102a, 102b) to reduce or fully eliminate a flow stasis, in accordance with an illustrative embodiment. The LVAD 100 may include some, or all of the multiple improved LVAD features described herein, including the stented inlet member 102 (shown as “Stented Inlet” 102a, 102b; see FIGS. 2A-2B), flexible rotor and/or pump housing 104 (FIGS. 3A-3B) and magnetic-based drive and bearing components (“maglev”) 106 (FIG. 6) (collectively shown as “Flexible Rotary and Magnetic Components” 104, 106), hydrophilic coating or surface 108 (see FIGS. 4A-4D), advanced controls and electronics 110 and advanced charging and energy management controls 112 (collectively shown as “Advanced Controls and Charging” 110, 112; see also FIG. 5), or a combination thereof.

In FIG. 1, the LVAD 100a includes a pump housing 114 forming a volume 115 (not shown; see FIGS. 3A, 3B). The pump housing 114 includes an inlet 116 and an outlet 118. The LVAD 100a includes a rotor 120 (e.g., flexible impeller; not shown, see FIG. 3A, 3B) disposed within the pump housing 110 to rotate in the volume 115 to pump blood. The LVAD 100a includes a motor 122 (not shown; see FIG. 4A, 4B) electrically coupled to the rotor 120 to drive rotation of the rotor 120. The stented inlet member 102 is coupled to the inlet 116 of the housing 114, the stented inlet member 102 having (i) an external structure 124 (e.g., fabric) configured to conform and contact an outer surface wall at a base of a left ventricle and (ii) an internal expandable body 126 configured to be placed in the chamber of the left ventricle to expand and contact the tissue wall in the chamber (e.g., to reduce risk of flow stasis after implantation). The output of the LVAD 100 is connected to an outflow graft 128 that couples to the aorta. External structure 124, as a fabric, may be sutured onto the apex of the left ventricle.

The internal expandable body 126 is configured to move between a stowed configuration (prior to being placed in the chamber, to be placed and extending into the left ventricle) and a deployed configuration (to be deployed after being placed in the chamber). The internal expandable body 126 when deployed form a fan out body that contacts the inner chamber/tissue of the left ventricle to avoid pocket of or reduce likelihood of flow stagnation in the chamber.

The structure of the internal expandable body 126, as well as its materials, in some embodiments, are configured able to absolvable over time, e.g., to be replaced by the heart tissue with only the neo-tissues being left behind after absolvation. In some embodiments, other biocompatible and biodegradable (e.g., absolvable) may be used. The internal expandable body 126 may terminate with a quick disconnect connector 129 to releasably and attachably connect the pump housing 114 during implantation and any subsequent surgeries. The quick disconnect connector can connect the pump housing 114 to the stented inlet member 102, so the LVAD 100 can be installed and swapped readily and capped off when necessary.

The advanced charger 110 operates with a subcutaneous implantable coil 132 that operates with an external coil 134 coupled to an external charger 136. The charger 136 can communicate with the LVAD 100 over a low-power communication channel and/or over power line communication through the charging circuit.

The magnetically levitated bearing drive system can reduce the wear of the bearing system to elongate the device life time and improve the overall device hemocompatibility. The system is configured to employ active control in the axial direction and passive control of radial position and inclination to minimize the number of control axes. Fewer control axes can decrease the outer diameter and the number of sensors and electronics. The maglev technology could also be applied to other direct blood-contact medical devices and/or devices have rotary components to reduce the induced hemolysis and the wear of the bearing system.

Deployment. To deploy the stented inlet 102, an opening can be made at the apex of the LV by a coring tool. Later, the crimped stent with the fabric and suture ring can be inserted into the left ventricle. After removing the crimper, the suture ring can be attached to the left ventricle using sutures. Lastly, the LVAD can be attached to the stented inlet using a quick disconnect connector.

Thrombosis risk reduction. Moving parts of the LVAD as well as formed stagnant flow locations in the heart can cause thrombosis formation. The thrombosis embolization can cause a high risk of ischemic stroke among LVAD patients, can block the inlet, causing pump failure. Blood stagnation points can trigger blood protein adsorption followed by a series of chain reactions, including thrombosis, ultimately leading to sepsis and death. A study observed the occurrence rate of thrombosis near the inlet annular to be as high as 96%. While prior LVAD designs have used, among other things, a sintered rough surface that promotes the formation of a biological neointima layer at the cannula, the anti-vWF antibody found in this layer was significantly reduced, but the thromboembolic problems were not eliminated.

The inflow cannula insertion angle and depth have been observed to affect the flow and the stagnation region around the cannula and in the pump and have been associated with thrombogenic events.

With respect to moving part damage, studies have shown that complications are associated with the blood damage caused by the supraphysiological shear stress in the LVAD during to the device's moving parts. When subject to high mechanical forces, blood cells can be damaged or destroyed, leading to hemolysis and activating platelets, which can lead to clotting and thrombosis. Moreover, high mechanical forces can break down the high-molecular-weight von Willebrand factor (vWF), a glycoprotein crucial in the blood coagulation process. The destruction of vWF has been associated with gastrointestinal bleeding after LVAD implantation. Reducing blood damage would improve hemocompatibility, thus improving the outcome of LVAD therapy.

The issues caused by moving parts and blood damage are not limited to LVADs but are also prevalent in other blood-circulating pumps, including extracorporeal membrane oxygenation (ECMO) and cardiopulmonary bypass (CPB) pumps. A promising way to treat uncontrolled hemorrhage, a cause of preventable death in the military, is to induce hypothermia using CPB. The lower temperature during CPB treatment puts the wounded soldier into a state of suspended animation, which can preserve the vital organs and neurological functions for an extended period of time for controlling the hemorrhage or transferring to capable medical facilities. To rapidly reduce the body temperature to 20° C., a high flow rate similar to the requirement of an LVAD is required for the pump. To this end, the improved mechanical and/or coating design can provide ultra-low thrombosis operation for other to other devices like CPB or ECMO.

Thrombosis due to quasi-static blood-material interactions. Thrombosis on surfaces due to the presence of whole blood can be elucidated as several events: protein adsorption, platelet adhesion/activation, leukocyte recruitment, and further activation of contact and complement pathways. Within seconds to minutes, blood serum proteins are adsorbed and undergo conformational changes on the surface. This layer of adsorbed proteins may allow subsequent adhesion and activation of nuclear platelets, which plays a role in the formation of the fibrin clot and recruitment of leukocytes (including monocytes and neutrophils). The platelets then initiate an inflammatory immune response and promote a cascade of events resulting in thrombosis inside the devices contacting blood. Surfaces that can eliminate unprecedented resistance to blood protein adsorption (the very first step of the blood coagulation cascade) can hinder the blood coagulation cascade, thereby providing favorable quasi-static blood-material interactions.

In several cases, the affinity of blood proteins to hydrophilic (i.e., water-loving) surfaces is lower than their affinity to hydrophobic (i.e., water-repellent) surfaces. Most physiological blood-contacting surfaces within the human body are hydrophilic, thereby reducing undesirable protein adsorption. Lower adsorption of blood proteins on hydrophilic surfaces compared to hydrophobic surfaces can be due to a combination of lower solid-liquid interfacial energy and the presence of a hydration layer. To reduce the solid-liquid interfacial energy (as nature prefers), blood proteins in the aqueous medium tend to adsorb at high energy solid-liquid interface of a hydrophobic surface rather than the low-energy solid-liquid interface of a hydrophilic surface. Consequently, hydrophilic surfaces can display lower blood protein adsorption than hydrophobic surfaces. Further, unlike hydrophobic surfaces, hydrophilic surfaces possess a hydration layer (i.e., water molecules bound to the surface), which must be displaced for protein adsorption. This presents an energy barrier, thereby preventing or reducing protein adsorption at short time scales (seconds to minutes).

### Example Stented Inlet Member

Referring to FIGS. 2A and 2B, two example LVAD 100a, 100b are shown with a stented or stent-like inlet member. FIG. 2A shows the LVAD 100b. As used herein, the term “stent” refers to a small, expandable tube that is insertable into the left ventricle to expand in the chamber of the left ventricle to contact tissue therein. The inlet member has or can have a structure similar to an arterial stent but expands to contact the tissue in the left ventricle to minimize the risk of flow stasis that could form around the interface between the LVAD and the left ventricle. In this instance, there is no blockage, as in the case of an artery or blood vessel.

To address eliminate the potential flow stasis and thrombosis and improve inflow hemodynamics, the exemplary LVAD 100a, 100b includes an internal, expandable body 126 having a fan out body that contacts the inner chamber/tissue of the left ventricle to avoid pocket of or reduce likelihood of flow stagnation in the chamber. In some embodiments, the internal, expandable body 126 is configured to move between a stowed configuration (prior to being placed in the chamber, to be placed and extending into the left ventricle) and a deployed configuration (to be deployed after being placed in the chamber). The internal, expandable body 126, in the deployed configuration, is defined by (i) a first section having a first circumference sized to couple to the inlet of the pump and (ii) a second section having a second circumference sized, wherein in the deployed configuration, to contact the inner surface wall/tissue of the left ventricle to eliminate flow stasis in the left ventricle. In the stowed configuration, the internal, expandable body 126 is defined by (i) the first section having the first circumference sized to couple to the inlet of the pump and (ii) the second section having the second circumference sized similar to first circumference.

As discussed above, in FIG. 1, and now in FIG. 2A, the LVAD 100a includes a pump housing 114 that includes an inlet 116 and an outlet 118. The stented inlet member 102a is coupled to the inlet 116 of the housing 114, the stented inlet member 102 has an external structure 124 configured to contact, and to some extent, conform to an outer surface wall 202 at the base of a left ventricle (e.g., LV apex). The stented inlet member 102 includes an internal, expandable body 126 configured to be placed in the chamber 203 of the left ventricle to expand and contact the tissue wall 205 in the chamber (e.g., to reduce the risk of flow stasis after implantation). In FIG. 2A, the tissue is shown to be partially transparent to provide a view of the internal expandable body 126 in an expanded configuration.

Inspired by the self- or balloon-expandable stent of transcatheter aortic valve technology, in some embodiments, the internal expandable body 126 includes a flexible mesh 204 that can be configured to be self-expanding or balloon-expandable. Self-expanding stents have interconnecting mesh elements, i.e., struts, that are configured to flexibly bend into a smaller configuration when placed under a force that can then expand circumferentially to an expanded position when deployed. Balloon-expandable have interconnecting mesh elements, i.e., struts, that are configured to be in a smaller configuration when not under force, but the struts are configured to stretch respect to one another to expand when subject to a force, i.e., via a balloon. The mesh 204 of the internal expandable body 126 can employ similar features as self-expanding or balloon-expandable, but the expanding features are (i) minimal at a proximal region 206 to the inlet 116 of the pump housing 114 and (ii) maximal at a distal region 208 of the device to form the fan out shape.

To this end, the internal expandable body 126, in the deployed configuration, is defined by (i) a first section 210 having a first circumference sized to couple to the inlet 116 of the pump housing 114 and (ii) a second section 212 having a second circumference sized, wherein in the deployed configuration, to contact the inner surface wall/tissue 205 of the left ventricle to eliminate flow stasis in the left ventricle. In the stowed configuration, the internal expandable body 126 is defined by (i) the first section having the first circumference sized to couple to the inlet of the pump and (ii) the second section having the second circumference sized similar to first circumference.

Flexible Mesh Pattern for Expansion or Compression. FIG. 2C shows an example pattern of the mesh 204, from FIGS. 2A and 2B, comprising a plurality of varying sinusoidal struts 214 (or hourglass struts), including two adjacent struts 214a, 214b, that are coupled to one another at peak portions for a sinusoid. In FIG. 2C, the varying sinusoidal struts have a constant periodicity, i.e., the distance between the peaks and trough of the sinusoid, and the longitudinal length 220 and amplitude/wide 222 of the peaks 216 of the sinusoid increases from the proximal region 206 to the distal region 208. FIG. 2D shows a mesh pattern, e.g., for mesh 204), of a transcatheter aortic valve having a set of generally triangular members 224. In the example shown in FIG. 2D, multiple triangular members 224 forms a strut section 226a that is linked, at linkages 228, to an adjacent structs section 226b. Each generally triangular member may include a notch to provide secondary structure expansion and compression. The mesh 204 may be manufactured by laser cutting Nitinol tubes as one example. Other manufacturing operations may be used.

Fabric. Referring to FIG. 2A, the internal, expandable body 126 includes a fabric 230 sized and shaped to the fan-out configuration. An example of the fabric 208 includes but is not limited to a polyester or a thermoplastic polymer resin (e.g., Polyethylene terephthalate (PET) (e.g., Dacron™)). The fabric 208 may be stretchable (or non-stretchable) and may surround the mesh 204, or the mesh 204 may be embedded within the fabric 204. Other fabric materials that may be used include polytetrafluoroethylene (e.g., expanded polytetrafluoroethylene (ePTFE)), biological tissue (e.g., transplanted pericardium tissue from human donors or animals), or a combination thereof.

The self- or balloon-expandable stent-like structure, e.g., surrounded with or embedded in the fabric (e.g., Dacron fabric) may be deployed into the left ventricle to replace the pump inlet cannula, e.g., employed in prior LVAD. The Dacron fabrics or other fabrics described herein may promote endothelialization that may cover the entire inlet member 102. Similar fabrics have been successfully implemented to prevent paravalvular leakage of transcatheter aortic valves. Absolved endothelium has been shown to provide a barrier against thrombus formation in relation to coronary stents, where it is observed that the risk of thrombosis is negatively correlated to the percentage of the struts covered by endothelium. The inflow to the LVAD may be smooth with minimal obstruction to generally conform to the inner tissue or chamber shape of the left ventricle.

Patient-specific design. In some embodiments, the inlet member 102, including the fabric and mesh pattern, may be fabricated as a patient-specific design for a given patient, e.g., to match a scan of the left ventricle of a patient. The scan may be computationally modeled, and a portion of the internal expandable body, e.g., flexible mesh, then 3D printed or laser cut to form a structure that can, once expanded, accommodate and/or conform to the complex anatomical variants of the ventricle for the patient.

In some embodiments, the computational modeling may include optimizing via finite element simulations, e.g., in Abaqus/CAE (manufactured by Dassault Systèmes). The mesh 204 may be manufactured by laser cutting Nitinol tubes.

### Example Flexible Rotor/Casing

FIGS. 3A-3G shows the flexible rotor and/or pump housing and design parameters for the exemplary LVAD 100a, 100b of FIG. 1 in accordance with an illustrative embodiment. Specifically, FIGS. 3A and 3B each show an example flexible rotor and/or pump housing. FIGS. 3C-3G shows design parameters.

In the example shown in FIG. 3A, the LVAD 100 (shown as 100c) includes a pump housing 114 (shown as 114a), forming a volume 115. The pump housing 114a includes an inlet 116 and an outlet 118. The LVAD 100a includes a flexible rotor 120 (shown as 120a) disposed within the pump housing 110 to rotate in the volume 115 to pump blood. The flexible rotor 120a includes a plurality of curved blades 302 formed of a polymeric material, e.g., to form an impeller. Other portions of the rotor may also be formed from the polymeric material. The curved blades 302 (shown as 302a) may be coated with or treated so the surface forms a slippery hydrophilic antithrombotic coating or surface 304 (see FIG. 3B). In the example shown in FIG. 3A, a portion of the surfaces may be coated with the slippery hydrophilic antithrombotic coating or treated as a slippery hydrophilic antithrombotic surface 304. In other embodiments, the entire internal surface of the pump housing 114 (not shown) may be coated with the slippery hydrophilic antithrombotic coating or treated as a slippery hydrophilic antithrombotic surface 304.

In FIG. 3A, the flexible rotor 120a includes a protrusion section 306 to engage with bearing 308 located in the internal portions of the pump housing 114a. The LVAD 100c includes a motor 122 shown having stator 122a and permanent magnet 122b. The permanent magnet 122b is fixably coupled to, or embedded in, the rotor 120a to drive rotation of the rotor 120a. A driver circuit 117 is embedded in the pump housing 114. The driver circuit 117 is electrically coupled to the stator 122a to drive the motor 122's rotation and adjust the motor 122's speed. The driver circuit 117 may couple to the sensors in the LVAD 100. The flexible rotor 120 and pump housing 114 may be configured with a maglev levitated driving system (see FIG. 6). Based on inviscid fluid dynamics theory, initial experimental and CFD tests have shown the pump can provide more than 90 mmHg of pressure at 5 L/min and 3000 RPM, which would be sufficient to support heart failure patients. FIG. 3D shows CFD results for the flexible rotor, the CFD results show good agreement with experiments (FIG. 3D).

FIG. 3C shows an example design 320 (based on a prior device, e.g., Abbott Labs' HeartMate 3™) and corresponding quantifications of the pressure and flow rate for comparison. The assembly of the device 320 is shown (FIG. 3C, subpanel A) and evaluated for different rotation speeds (FIG. 3C, subpanel B). The model 320 was designed based on proven industrial design strategies. The device 320 was driven by an external shaft and evaluated for flow performance and flow characteristics.

The blade of the rotor 120 can be made of cast polyurethane rubber (e.g., for durability and chemical stability). The rotor 120 can also be made of other flexible materials and other methods, such as injection molding. The acrylic casing can be coated with a layer of the same material to make it flexible as well. The rotor blades have a wide range of hardness, for example, from Shore 10A to 100A. Flexible water-clear polyurethanes (BJB Enterprise, Tustin, CA) 22 may be used for the rotor. The flexible rotor and casing may be coated with the antithrombotic coating.

In FIG. 3D, the flow field and hemolysis index (HI) distributions for two rotor designs (“10-30” design and “70-70”) are shown. The “10-30” and “70-70” designs are optimized for flow, though not yet optimized for blood damage. The in-plane flow vectors are shown in FIG. 3D, subpanel A and C, and the hemolysis index contour is shown in FIG. 3D, subpanel B and D. FIG. 3D shows a comparison of the two rotor designs for flow field to compare the two rotor designs for the hemolysis index under a same supporting flow rate and head rise for different RPMs.

The hemolysis index measures the ratio of free hemoglobin over total hemoglobin and can be obtained by an Eulerian hemolysis model described in Yu et al., The difference between the two rotors is the leading and trailing edge blade angles, measured between the blade chordwise and the circumferential direction. The rotor “10-30” is the optimized design for the flow, having a leading-edge angle of 10° and a trailing-edge angle of 30°. Indeed, the flow vectors follow the blade, unlike the large-scale recirculation zones seen for the 70-70 rotor. However, when comparing the blood damage measured at the exit of the LVAD model, the 70-70 rotor shows a 5% lower hemolysis index than the 10-30 rotor.

The less-than-optimal flow field of the “70-70” rotor can provide less blood damage. With a straighter blade, the pump can operate at a much lower RPM. To generate the same head and flow, 3163 RPM was employed for the 10-30 rotor, while the 70-70 only needs to operate at 2749 RPM. Lower RPM may cause less shear on the blood, thus reducing the blood damage. The shorter blades of the “70-70” rotor may also provide a smaller blood-contacting surface that may reduce blood damage. Notably, the results of FIG. 3D suggest there exists an optimal point to provide flow while minimizing blood damage. FIG. 3E shows the hemolysis index as a function of number of blades (subpanel A) and pump size (subpanel B).

Flexible rotor performance. FIG. 3F shows the efficiency performance of the flexible rotor blades as compared to rigid rotor blades. The data shows the flexible rotor with the flexible rotor blades can increase the power production efficiency. FIG. 3F, subpanel A shows a four-piece mold for the rotor adapted from MacPhee and Beyene. Several fluid dynamic studies have shown that flexible wings or blades may offer higher performances to aircraft and turbomachines, especially under off-design conditions [13′]-[15′]. Due to fluid structure interactions of the wing and flow field, flexible rotor blades have been found to adapt to the surrounding flow, leading to efficiency increases in turbomachines [16′]. The results of FIG. 3F indicate that the flexible rotor blades can increase the flow efficiency of LVAD 100.

FIG. 3G shows an example design for an impeller and corresponding geometric parameters for the exemplary LVAD (e.g., 100). In FIG. 3G, the geometry of the pump is characterized by nine parameters: inlet diameter (D1), rotor diameter (D2), leading-edge angle (β1), trailing edge angle (β2), number of blades (z), blade thickness (d), blade tip gap size (distance between the blade tip and cutwater) (h), blade wrap angle (φ), and outlet diameter (D3).

Machine learning optimization. Machine learning can be used to optimize the LVAD design, e.g., of FIG. 3G. The program may be developed in MATLAB to generate the LVAD model based on these parameters. Subsequently, 3D geometries can be built in Solidworks. The Hemolysis Index (HI), a measure for blood damage, may be optimized at the design condition of 5 L/min and 90 mmHg. HI can be determined as the function of all the parameters in question: HI=function (RPM, D1, D2, β1, β2, z, d, h, φ, D3) and evaluated using Computational Fluid Dynamics (CFD).

The analysis may start with simulating about 500 cases as the training dataset. A separate 200 cases may be randomly generated and used to validate the model. The process may be automated in Matlab and Ansys Workbench. A deep neural network (DNN) architecture may be used to predict HI based on the ten input parameters. The DNN may be trained using the training dataset and evaluated using the validation set. After the model is accurately trained, stochastic gradient descent may be used to predict the combination of parameters that has the lowest possible HI. The efficiency may be optimized using a similar process.

Other studies have shown that flexible wings can suppress laminar separation bubbles which can be a cause of performance degradation at a low Reynolds number [17′]. Such flexible rotor concepts have been used in designing a foldable transcatheter VAD [18′]-[20′] and a flexible pump [21′], [22′] to provide superior flow efficiency performance to their ridged counterparts at certain flow regimes [22′]. Shear stress reduction was not indicated as a consideration in the study.

Considering the complicated inflow conditions inside a patient's heart, an LVAD may never operate under the optimal flow design condition. Blood damage may rise significantly under complex real-world pulsatile conditions. The exemplary LVAD (e.g., 100) in employing flexible rotors may reduce blood damage by adapting to the complex inflow conditions of the heart.

### Slippery Hydrophilic Antithrombotic Coatings for LVADs

FIG. 4A shows a slippery hydrophilic (SLIC) surface that can counter intuitively spread aqueous droplets rather than allowing water to stick as conventional hydrophilic surfaces. While conventional hydrophilic surfaces can reduce blood protein adsorption on short time scales, at longer time scales (minutes to an hour), blood proteins can adopt hydrophilic conformations to diffuse through the hydration layer and then adsorb on the surface. Because water molecules within the hydration layers are disordered and loosely bound to conventional hydrophilic surfaces, blood proteins can penetrate through the hydration layer and their irreversible adsorption, as shown in FIG. 4A.

On the exemplary SLIC surfaces, water molecules within the hydration layers are highly ordered and tightly bound to the surface, thereby forming “ice-like” hydration layers as shown in FIG. 4B. Prior molecular dynamics (MD) simulations have indicated that ice-like hydration layers can reduce friction on the molecular scale, allowing bulk water molecules to slip or slide tangentially to the surfaces with ice-like hydration layers. The high tangential mobility of water molecules in the vicinity of the ice-like hydration layer can impart substantial lateral forces on adsorbed blood proteins, as indicated by arrows in FIG. 4B, subpanel B. These lateral forces are expected to dislodge adsorbed blood proteins and hinder the subsequent steps (e.g., platelet adhesion and activation, leukocyte adhesion, etc.) of the blood clotting cascade, as shown in FIG. 4C, subpanel C. Indeed, results of a conducted study indicate that blood proteins do not adsorb on the exemplary SLIC coatings after a week. In contrast, the state-of-the-art coatings may prevent protein adsorption for just a few minutes to a few hours, making the exemplary SLIC coatings a breakthrough in surface science.

FIG. 4B shows the anti-thrombotic properties of SLIC coatings. In FIG. 4B, subpanel A, a schematic depicting disordered hydration layers on conventional hydrophilic surfaces is shown that results in high protein adsorption. Subpanel B shows a schematic depicting ice-like hydration layers on SLIC coatings, resulting in negligible protein adsorption. Subpanel C shows water droplets unable to slide on conventional hydrophilic surfaces. Subpanel D shows water droplets sliding on SLIC coatings at a low tilt angle.

FIG. 4C, subpanels A and B, shows schematics depicting fully developed blood flow between the walls of a conventional hydrophilic surface and an SLIC coating, respectively. FIG. 4C, subpanel C shows a schematic depicting the anti-thrombotic nature of SLIC coatings on LVADs. Additional examples and descriptions of the hydrophilic coating may be found in published PCT application no. WO2019074791A1, which is incorporated by reference herein its entirety.

Thrombosis due to dynamic blood-material interactions. Thrombosis due to dynamic flow conditions in LVADs can arise from supraphysiological shear stress and flow stagnation in the device, which can lead to hemolysis, platelet activation, breakdown of von Willebrand factor (vWF), etc. As blood flows past conventional LVAD surfaces, there is no slip between blood and a conventional LVAD surface. In other words, a no-slip boundary condition, when it exists at the blood-material interface, can increase blood stagnation and shear stresses. LVAD surfaces with SLIC coatings, which induce slipperiness at the blood-material interface due to ice-like hydration layers, can reduce friction. As blood flows passed such LVAD surfaces with the SLIC coatings, blood stagnation and shear stresses are reduced, thereby reducing hemolysis, platelet activation, breakdown of vWF, etc., and reducing thrombosis.

Consider a steady, fully developed blood flow through a conduit without SLIC coatings and with SLIC coatings. Without the SLIC coatings, conventional hydrophilic surfaces can result in a no-slip boundary condition at the wall, i.e., the velocity of blood in contact with the wall, U_{no SLIC}=0, resulting in significant flow stagnation. Furthermore, the no-slip boundary condition results in higher velocity gradients (dU/dr)_{no SLIC} at the wall (FIG. 4C, subpanel A), which in turn can lead to higher shear stresses τ_{no SLIC}∝(dU/dr)_{no SLIC} at the wall. Such higher shear stresses can result in hemolysis, platelet activation, breakdown of vWF etc., resulting in thrombosis. In contrast, the slipperiness induced by the SLIC coatings results in a finite slip at the solid-liquid interface, i.e., the velocity of blood in contact with the wall, U_{SLIC}>0 (finite) (FIG. 4C, subpanel B), resulting in reduced effective interfacial area for flow stagnation. The finite slip results in lower velocity gradients (dU/dr)_{SLIC} at the wall, which in turn leads to lower shear stresses τ_{SLIC}∝(dU/dr)_{SLIC} at the wall. Such lower shear stresses reduce hemolysis, platelet activation, breakdown of vWF etc., and can result in improved dynamic blood-material interactions and reduced thrombosis. FIG. 4C, subpanel C shows exemplary SLIC coating having superior hemodynamics can reduce thrombosis.

SLIC coatings design. SLIC coatings with ice-like hydration layers can improve both the quasi-static and dynamic interactions with blood, thereby reducing thrombosis in LVADs. The SLIC coating can have both hydrophilicity and slipperiness.

Hydrophilicity: the fundamental macroscopic measure of the wettability of a surface is the equilibrium contact angle θ. On a non-textured (or smooth) surface, the equilibrium contact angle θ for any contacting liquid may be given by Young's equation as: γ_{LV} cos θ=γ_{SV}−γ_{SL}. Based on the equilibrium contact angle θ of a water droplet, a surface may be classified as hydrophilic when θ<90° and hydrophobic when θ>90°. Young's equation shows that high solid surface energy γ_{SV} results in low equilibrium contact angles. Consequently, surface chemistries with high solid surface energy (e.g., PEG groups, ionic groups, zwitterionic groups, etc.) are logical choices for designing hydrophilic surfaces.

Slipperiness: the fundamental macroscopic measure of slipperiness is the contact angle hysteresis Δθ, which is defined as the difference between the advancing θ_{adv} and the receding θ_{rec} contact angles on a solid surface (i.e., Δθ=θ_{adv}−θ_{rec}). Contact angle hysteresis Δθ can arise from surface chemical inhomogeneity and physical inhomogeneity. Physically, contact angle hysteresis is a measure of the energy dissipated during the motion of a liquid droplet along a solid surface. When a surface displays very high contact angle hysteresis (regardless of the equilibrium contact angle), a liquid droplet can be immobile on the surface (FIG. 4B, subpanel C). On the other hand, when a surface displays very low contact angle hysteresis (regardless of the equilibrium contact angle), a liquid droplet can slide on the surface. Consequently, low contact angle hysteresis can lead to low sliding angle ω (i.e., the minimum angle by which the surface should be tilted for the droplet to slide (FIG. 4B, subpanel D). Based on the balance between the work done by gravity and the energy dissipated by adhesion, the sliding angle on a smooth solid surface can be given as ρVg sin ω≈γ_{LV} D_{TCL}(cos θ_{rec}−cos θ_{adv}), where ρ and V are density and volume of the liquid droplet, respectively, g is the acceleration due to gravity, and D_{TCL} is the width of the triple phase contact line perpendicular to the sliding direction. The sliding angle ω is an inverse measure of the slipperiness of a liquid droplet on a solid surface. Slippery surfaces with low sliding angles and low contact angle hysteresis may be designed with surfaces that have low physical inhomogeneity (i.e., smooth surface) and low chemical inhomogeneity (i.e., high grafting density). Smooth surfaces with ultra-high grafting density are ideal for designing slippery surfaces.

Although conventional hydrophilic surfaces can display low water contact angles due to their high solid surface energy γ_{SV}, they can display high adhesion to the surface due to high contact angle hysteresis. On a macroscopic scale, the high adhesion can manifest as droplets not being able to slide past the surface, even on a vertical surface, making the surface non-slippery. The exemplary SLIC surfaces are counter intuitive as they are not only hydrophilic but also have very low sliding angles and contact angle hysteresis, indicating high slipperiness. Recent molecular dynamics (MD) simulations showed that slipperiness and low adhesion may emerge in hydrophilic surfaces with ice-like hydration layers with strong hydrogen-bonding that may not be disturbed by any protein conformations. Such ice-like hydration may be achieved on smooth surfaces with high grafting density of hydrophilic molecules on the surface. Ice-like hydration layers result in high mobility of the liquid in the vicinity of the solid-liquid interface.

The exemplary SLIC coatings with ice-like hydration layers can result in high mobility of the liquid molecules in the vicinity of the solid-liquid interface. On a macroscopic scale, ice-like hydration layers manifest as aqueous droplets or blood droplets sliding past surfaces at low sliding angles, indicating slipperiness. On a microscopic scale, ice-like hydration layers can manifest as finite slip length. On a molecular scale, ice-like hydration layers can manifest as high lateral mobility of water molecules, imparting substantial lateral forces to dislodge adsorbed blood proteins.

Fabrication and characterization of SLIC coatings. SLIC coatings may be fabricated on acrylic and polyurethane substrates via consideration for surface activation parameters, molecular architecture and reaction coordinates. Acrylics and polyurethanes are used in prior LVADs and are amenable to future additive manufacturing techniques. However, the application of SLIC coatings is not limited to the abovementioned substrates. Further optimizing the exemplary SLIC coatings (i.e., achieving lower physical and chemical inhomogeneities) for a maximum antithrombotic response may be carried out by systematically tailoring the hydroxylation parameters, molecular architecture, and synthesis reaction coordinates. The optimized SLIC coating protocol may then be seamlessly transferred to efficiently coat the blood-contacting surfaces of the exemplary LVAD designs.

The surface of LVAD polymers (i.e., acrylic and polyurethane) may be modified via liquid-phase silanization, [24′], [25′] because it is a simple, versatile, and scalable technique and a wide variety of organosilanes with hydrophilic chemistries are commercially available.

FIG. 4D shows organosilanes consisting of two chemical moieties-functional organic groups and reactive groups. The functional organic groups may include non-reactive moieties (e.g., hydrophilic chemistries like PEG, ionic or zwitterionic oligomers, etc.), which can impart the desired characteristics (e.g., hydrophilicity) to the substrate. The reactive groups may include hydrolyzable moieties (e.g., chloro, amino, methoxy, ethoxy, etc.), which react with the underlying hydroxylated substrate (i.e., surface with —OH groups) to form a durable covalent siloxane bond. The degree of silanization (or grafting density of the functional groups) on a surface depends on the hydroxylation of the substrate, the molecular architecture of the silane [26′]-[28′], and silanization reaction conditions (e.g., type of solvent, silane concentration, type of catalyst, catalyst concentration, silanization time, water concentration, temperature, pH, etc.) [24′], [25′], [29′]-[31′].

In some embodiments, the hydroxylation time of 60 seconds to 3600 seconds may be used in oxygen pressure (1 psi to 30 psi), with an oxygen flow rate (1 cc/min to 50 cc/min) and plasma power (1 watt to 50 watts), followed by silanization to graft brushes of different hydrophilic chemistries.

Influence of hydroxylation: Substrates can have a hydroxyl (—OH) groups on the surface for silanes to react with them [24′], [25′]; the high degree of substrate hydroxylation can lead to a higher degree of silanization or higher grafting density, which can ensure high chemical homogeneity that, in turn, is a design requirement for SLIC coatings [28′], [32′], [33′]. To achieve a high degree of hydroxylation, the LVAD polymer surface (e.g., made of acrylic and polyurethane) can be modified using oxygen plasma. Oxygen plasma for hydroxylation is straightforward, portable, and scalable. While too low of an oxygen plasma exposure results in insufficient hydroxylation, too high of an oxygen plasma exposure can lead to substrate deterioration or roughening (i.e., physical inhomogeneities), [34′]-[38′], which is undesirable for designing SLIC coatings. The oxygen plasma parameters (e.g., hydroxylation time, oxygen pressure, oxygen flow rate, plasma power, etc.) can be used to obtain the desired degree of substrate hydroxylation. The hydroxylated substrates may be modified with silanes [42′]-[52′].

Influence of molecular architecture: The molecular architecture of the silane (i.e., the type and length of functional groups, the type and number of reactive groups, etc.) may influence the grafting density of the silane on the substrate [25′]-[28′]. To obtain the high grafting densities necessary for designing SLIC coatings, the molecular architecture can be tuned for grafting density, which can determine the effectiveness of SLIC coatings, which in turn determines their antithrombotic nature. While smaller molecular sizes and chain lengths (i.e., number of repeating units in the oligomer or length of the molecule) of functional groups (say, PEG, ionic or zwitterionic) and higher number and reactivity of the reactive groups can allow higher grafting density (i.e., lower chemical inhomogeneity), larger molecular sizes and chain lengths of the functional group and higher number and reactivity of the reactive groups leads to lower physical inhomogeneity [44′], [48′], [53′]-[56′]. By systematically tuning the molecular architecture, the optimum combination of low physical and chemical inhomogeneities can be determined and, consequently, an effective SLIC coating.

Influence of synthesis reaction coordinates: While silanization is a straightforward technique, it is also a versatile technique providing many degrees of adjustments to turn to vary the synthesis reaction coordinates (e.g., type of solvent, silane concentration, type of catalyst, catalyst concentration, silanization time, water concentration, temperature, pH, etc.) to obtain effective SLIC coatings [24′], [25′], [28′], [57′].

### Wireless Energy Transfer and Communication

FIG. 5 shows an example charger 136 (shown as a wireless energy transfer and communication system 136a) that can be implemented in the exemplary Centrifugal LVAD. The wireless energy transfer and communication system 136a can reduce infection risk, enable free movement of the patient, and improve the quality of life.

The wireless energy transfer and communication system 136a includes an external power source for the SLIC LVAD pump. The rechargeable energy storage device in the implanted controller is designed to provide uninterrupted power when the external wearable power source (e.g., provided in a belt or vest) is removed for short periods of time. The communication link can provide feedback to the external power system for the system monitoring of electrophysiology and improved control of the charging circuit to reduce heating and improve both mechanical and electrical cardiac function.

Wireless Energy Transfer. The LVAD 100 (e.g., 100d) may employ an RF wireless energy transfer subsystem that operates with an external rechargeable device, coupled to a belt or vest, to reliability deliver and charge its energy storage. The external rechargeable device can inductively charge the LVAD through an implanted internal antenna. The implanted charging components include circuitry to provide power to the LVAD and charge an internal energy storage device containing a combination of rechargeable batteries, supercapacitors, and hybrid supercapacitors for periods when the external belt or vest is removed and/or not available.

The charging subsystem may include communication between the implanted LVAD 100 and other internal sensors and the external vest or belt controller for system monitoring and/or feedback control. In some embodiments, the communication may be based on low-energy Bluetooth, or other communication described herein, to collect and send sensor readings, e.g., ECG data, to the charging subsystem. The antenna may include a backplane material to aid in focusing energy transmission. The antenna may additionally include temperature and position sensors to monitor coupling efficiency. Antennas may be built with flexible materials to conform to body shapes to also improve comfort.

Control algorithms for improved dynamic LVAD performance. The wireless energy and communication may serve as the controller for the LVAD. Calibration values may be modified during operations, e.g., via ML controls, to react to different dynamic physical loading events. In combination with the modified LVAD calibration, control algorithms may pulse the transmitted RF energy to provide sufficient power to the SLIC LVAD to monitor and reduce tissue heating.

In FIG. 5, the charger 112 (shown as 112a) includes a rechargeable external power source 136, transmitter coil 134, and receiver coil 132 to power an internal rechargeable energy source 502 (shown as “Implanted Receiver” 502) containing rechargeable batteries and hybrid supercapacitors 504 (shown as “energy storage”) to power the implanted LVAD 100 (shown as 100d). In some embodiments, the internal energy source may power (e.g., 5 W) the LVAD motor and magnetic levitation-bearing systems for at least twenty minutes.

In the example shown in FIG. 5, the external transmitter system 136 includes a microcontroller 506 (e.g., an ARM or a Xilinx Zynq FPGA). An oscillator 508 may be used to create an alternating current (AC) signal that is provided to a digital upconverter circuit 510 to create a radio frequency (RF) signal. The RF signal may be routed to a power amplifier (PA) (also shown in 510). The output of the PA 510 may be then connected to the transmit coil antenna 134 through an electrical matching network to ensure proper signal integrity. The power level can be adjusted to provide sufficient power for the implanted controller 502, LVAD (100d), and rechargeable energy storage device 504. The controller 506 may adjust the power delivery based on feedback from the implanted controller. The external transmitter system 136 includes rechargeable batteries 512 and may be packaged to be coupled to a waist belt or vest, e.g., for close placement to the implanted receiver coil 132.

The LVAD 100d may include an implanted internal electrical drive lead line 514 that connects the receiver coil 132 to the control unit and associated circuitry. The drive line 514 may follow along the path under the skin. The implanted control unit 502 may be similar in size and shape to a pacemaker control unit and placed with the receiver antenna 132 under the skin. The implanted control unit 502 includes the antenna coil 132 to capture the energy through inductive coupling.

The implanted control unit 502 may include rectifier circuits 516 to convert the received RF alternating current signal into a direct current (DC) signal, to power an internal low-power control processor 518 (e.g., Texas Instruments MSP430 or an application-specific integrated circuit (ASIC) chip). The processor 518 may be connected to the controller 520 configured to execute the charging operation for the internal rechargeable energy storage device 504.

One or more rechargeable battery and energy storage components may be used, e.g., super-capacitor or hybrid super-capacitor. Super-Capacitors are rechargeable energy storage devices that are capable of significantly more charge/recharge cycles than rechargeable batteries. The processor 518 may interface with the motor control and magnetic levitation control electronics (e.g., also in controller 520). The processor 518 may connect to a communications module 522 (shown as “LP Comm” 522) to transmit pump performance and power delivery data to the external power transmitter. The communication module 522, as an internally implanted transmitter, such as a low-energy Bluetooth link, can transmit LVAD status information along with ECG data to the external controller to improve control algorithms and for long-term electrophysiology monitoring. The communication module 522 may employ other communication protocols, e.g., Bluetooth, ZigBee, and Z-Wave communications or a power line carrier (PLC) communication protocol.

Both the internal control unit 502 and the external power source 136 may each have integrated position and temperature sensors and corresponding circuitries to monitor heat due to antenna misalignment and the inefficiencies of wireless energy transfer. The controller 520 may include control software to allow for the pairing of the modules and for the transfer of data from the various internal sensors. The Bluetooth module 524 (shown as “LP Comm” 524) at the external power source may be additionally paired with a smartphone for monitoring of the LVAD 100d (e.g., by both the patient and the cardiologist).

Electrophysiology analysis to control LVAD function. Electroanatomical mapping may be performed before and after LVAD implantation to establish a baseline for comparison, assess acute changes, and assess longer-term outcomes in changes to the native conduction patterns. Additionally, a 12-lead surface ECG may be utilized for global conduction assessment. The broad range of electroanatomical conduction data across various time points and critical cardiovascular events can offer much-needed insight into longer-term patient management. This level of understanding of conduction before and after LVAD implantation can offer clues about whether additional rhythm management, such as ablation, could benefit patients in the long term.

The electronic control and LVAD pump motor can create unwanted electrical noise that can affect the sensors employed for monitoring ECG and pacing. In addition to shielding, active DSP noise cancellation algorithms may be used to improve signal quality. Machine learning (ML) algorithms (e.g., executing at the charger 136 or cloud infrastructure coupled through the charger 136) may be developed to model LVAD time-series data and outlier events.

LVAD Control. LVAD control algorithms may include a trained machine-learning model configured to adapt LVAD motor operation to dynamic loads due to patient activity and stress. The algorithms may also monitor the electrical conduction of the heart and modify LVAD operation to allow for left ventricle rest to enable healing and improved heart function.

The control algorithms may adjust LVAD operation to reduce energy consumption in periods of reduced need as when at rest. Communication data rates from the internal controller and sensors to the external controller and vest can also be reduced during periods of reduced activity. Reduced load on the LVAD system also reduces the need for wireless energy transfer to maintain the charge of the internal rechargeable battery and hybrid super-capacitor.

LVAD Drive System Design with Magnetic Levitation

FIG. 6 shows an example LVAD 100 (shown as 100e) configured with a drive system (also referred to as a “maglev system”) with magnetic levitation comprising a rotor 120 (shown as “impeller” 120b) having an attached rotor permanent magnets 602, a stator 604 embedded within the pump housing 114 adjacent to the inlet member 102, and an active magnetic bearing 606 (shown as “MagLev Bearing” 606) located at the bottom of the pump housing. The pump housing 114 has a position sensor 608 (shown as “eddy current sensor” 608) configured to monitor the gap between the impeller tip and the rotor 120b. The drive system includes an axial magnetic bearing and radial passive magnetic bearing with an independent BLDC motor to prevent rotor touchdown radially and adjust rotor position axially in response to changes in force during normal and extreme conditions. The drive system can perform active control in the axial direction and passive control of radial position and inclination. Fewer control axes may decrease the outer diameter and the number of sensors and electronics needed. In FIG. 6, the impeller 120b is fixably attached to the rotor's permanent magnets 602. Inside of the LVAD housing, the stator 604 is embedded adjacent to the inlet cannula 116.

The active magnetic levitated bearing 606 located at the bottom of the LVAD with eddy current sensor 608 can monitor the gap between the impeller tip to the maglev. The eddy current sensor 608 may be analyzed to improve the control of LVAD pump speed. In the radial direction, the rotor permanent magnets 602 lines up with the stator 604, and in the axial direction, the maglev bearing 606 resists the force from the electromagnetic force between the motor stator 604 and rotor 120b and the rotor force due to gravity. In some embodiments, the eddy current sensor 608 is coupled to the electronics 117 to operate the control of LVAD pump speed based on the sensor reading.

When implanted in a large animal model, the SLIC LVAD may contribute up to 5 L/min of the total cardiac output for the calve model and may maintain physiological arterial pressure and low hemolysis rate.

Additional examples and description of the LVAD Drive System with Magnetic Levitation may be found in published PCT application no. WO2023189970A1, which is incorporated by reference herein its entirety.

### EXPERIMENTAL RESULTS AND ADDITIONAL EXAMPLES

Several studies have been conducted on various features of the LVAD system described herein. A study was conducted to evaluate the inlet member design, flexible blade and casing, SLIC coating,

### Inlet Member Study

A design was computationally modeled and optimized via finite element simulations, e.g., in Abaqus/CAE (manufactured by Dassault Systèmes). The mesh 204 was manufactured by laser cutting Nitinol tubes. After suturing the Dacron fabric to the stent, the inlet member 102 was deployed into a patient-specific silicone left ventricle model and mounted to a left heart simulator flow loop. Three-dimensional particle tracking velocimetry was used to thoroughly evaluate the flow and blood damage potential in the stented inlet and in the LVAD model. Results may be compared with the same model with a straight inlet cannula, to confirm that the stented inlet does eliminate the stagnation regions around the inlet and thus reduce the thrombosis risk completely. The test condition ranged from a healthy beating heart to a pathological failing heart.

### Heartbeat Synchronized Speed Modulation for Pediatric LVADs

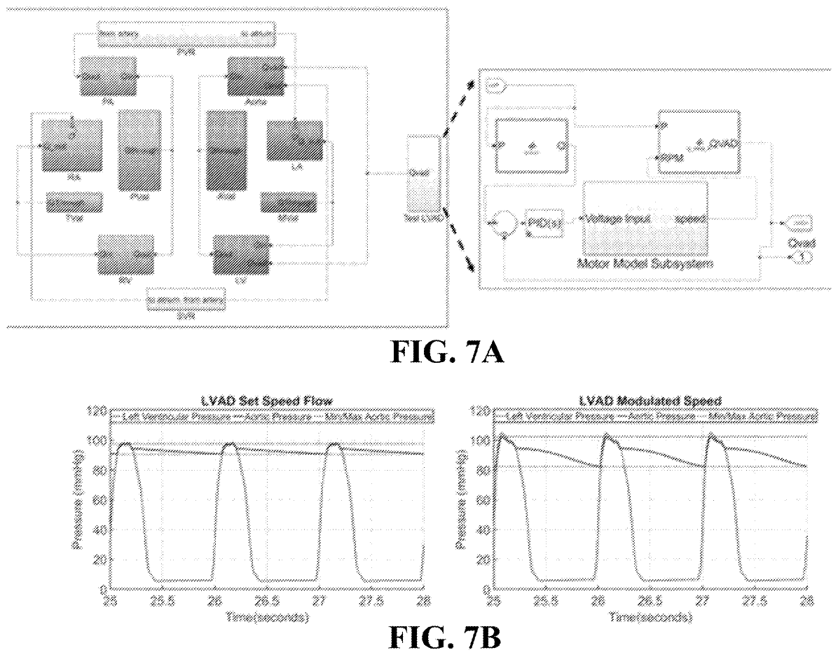

Increasing the pulsatility of combined LVAD and heart output may be done by modulating the speed of the pump driver in synchrony with the heart rhythm. Increased pulsatility can be closer to the physiological norm, can lessen blood stagnation, and can improve baroreceptor feedback for pressure regulation. FIG. 7A shows a previously validated computational model of a mock circulatory loop that can be used as a platform to observe the hemodynamic effects of synchronized pump speed modulation on the heart. The study established a model of a brushless DC motor driving the pediatric LVAD using the speed, torque, and inertia constants of the manufacturer specification sheet to represent the motor's ability to change speed. The pressure and flow HQ curve of the LVAD impeller is used to calculate the pump output flow at different pressures and impeller speeds. The flow of the set speed LVAD is 3 L/min, and the flow of the speed-modulated LVAD varies between 1 L/min and 5 L/min at diastole and systole, respectively. The study used a PID controller to control the pump speed to reach the desired flow output.

FIG. 7B shows the left ventricular and aortic pressure controlled by a set speed LVAD (subpanel A) and a speed-modulated LVAD (subpanel B). The modulated LVAD flow resulted in larger changes in aortic pressure. The speed modulated LVAD had an aortic pressure difference of 20 mmHg between the minimum diastole and maximum systole values, while the set speed LVAD had a difference of 7 mmHg. The set speed LVAD and the speed modulated LVAD had similar average aortic pressure values of 93 mmHg and 91 mmHg, respectively. The changes in aortic pressure at systole and diastole resulting from the speed-modulated flow show that synchronized motor speed modulation results in physiologic conditions closer to the norm.

### Flexible Blade and Casing Evaluation

The study additionally evaluates the integration of the flexible blade and casing into the LVAD design. In the study, the blade was made of cast polyurethane rubber for its durability and chemical stability. The acrylic casing was coated with a layer of the same material. The study developed the exemplary LVAD with acrylics for its low cost, hemocompatibility, and amenable to potential additive manufacturing techniques. Moreover, polyurethane rubber can be readily coated with SLIC coatings. The performance and blood loop test may be carried out to evaluate the reduction in blood damage by different material hardness. The best ones were evaluated using particle image velocimetry. The deformation of the blade and casing was measured under different flow conditions. The durability of the material was thoroughly evaluated.

Flexible Rotor Design with Machine Learning. The LVAD design was optimized using machine learning. Based on the industry-proven design concept [23′], the geometry of the pump was characterized by nine parameters. As shown in FIG. 3G, the parameters that control the geometry and their ranges are: inlet diameter (D₁=10-15 mm), rotor diameter (D₂=20-45 mm), leading-edge angle (β₁=5-90°), trailing edge angle (β₂=5-90°), number of blades (z=3-6), blade thickness (d=1-4 mm), blade tip gap size (distance between the blade tip and cutwater) (h=1-4 mm), blade wrap angle (φ=10°-600°), and outlet diameter (D₃=10-20 mm). The study developed a program in MATLAB to generate the LVAD model based on these parameters. Subsequently, 3D geometries were built in Solidworks. The Hemolysis Index (HI), a measure for blood damage, was optimized at the design condition of 5 L/min and 90 mmHg. HI was determined as the function of all the parameters in question: HI=f(RPM, D₁, D₂, β₁, β₂, z, d, h, φ, D₃), and was evaluated using Computational Fluid Dynamics (CFD). The study started by simulating about 500 cases as the training dataset, covering all the extremes of the parameters. A separate 200 cases were randomly generated and used to validate the model. The whole process was automated in Matlab and Ansys Workbench.

The study utilized a deep neural network (DNN) architecture to predict HI based on the ten input parameters. The DNN was trained using the training dataset and evaluated using the validation set. After the model was trained, another machine learning algorithm (stochastic gradient descent) was used to predict the combination of parameters that has the lowest possible HI. The efficiency was optimized using a similar process as well.

Numerical simulations. Using Ansys Fluent 3D pump models, the study simulated supporting flow conditions of 90 mmHg and 5 L/min. The rotor speed were adjusted accordingly to match this condition. Performance data from the in vitro experiments were used to validate the CFD simulations, and high-resolution PIV experiments were used to calibrate the turbulence models. A stress and exposure time-based hemolysis model was applied to assess blood damage. The parameters of the hemolysis model were calibrated using preliminary blood loop experiments.

Off-Design Point Operations for LVADs using flexible rotor blades. The operating speeds of the LVAD are often tuned to accommodate patient variability which might lead to operating the LVAD at off-design points, potentially causing a loss in efficiency and an increased risk of thrombosis/hemolysis. The flexible rotor blades may deform in response to the complex inflow conditions and, with an appropriate geometry and flexibility, generate a flatter performance curve.

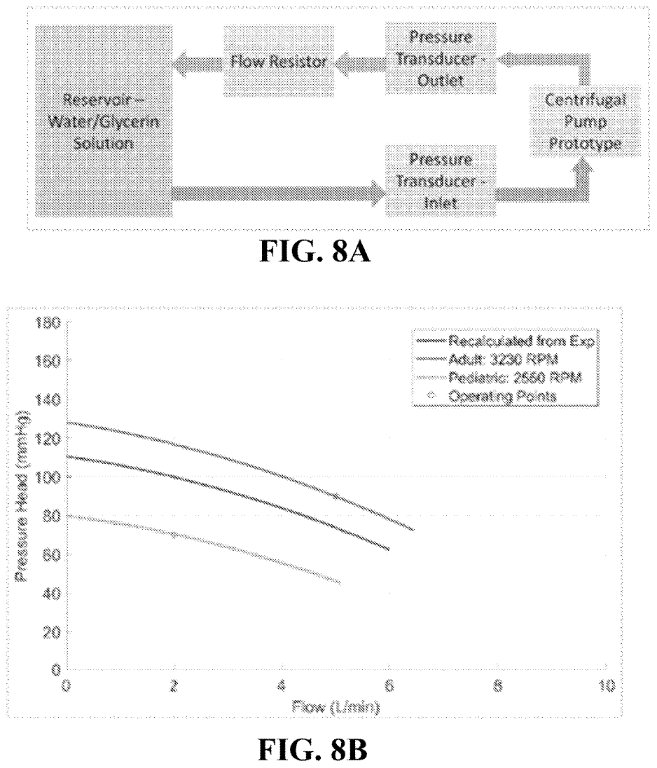

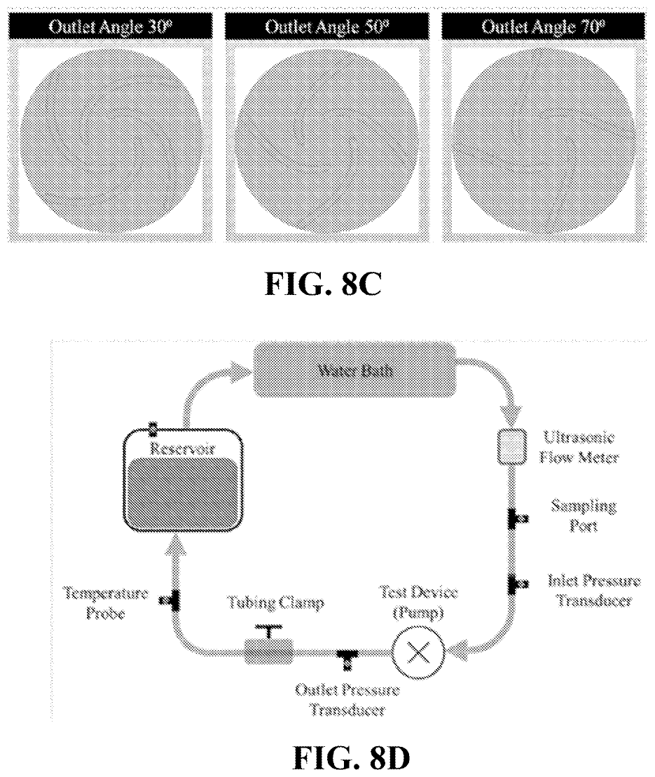

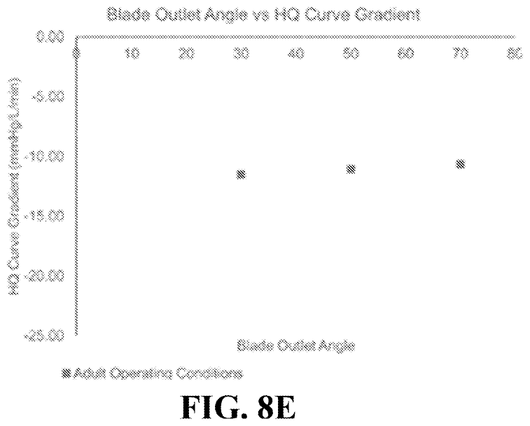

The study experimented with the hydraulic performance of varied impeller geometries for an LVAD with flexible rotor blades at pediatric and adult pressure/flow conditions. FIG. 8A shows a schematic of a hydrodynamic test setup employed to evaluate the hydraulic performance of the rotor and impeller. The study manufactured the flexible rotor blades by casting polyurethane resin (Easton, PA) of shore hardness 60A in a four-piece mold. The study 3D-printed the mold pieces using a J850 Pro (Stratasys Ltd, MN, USA). The inlet angle of the blades was set at 20° and the outlet angle was varied from 10° to 70°. The blade thickness was 1.2 mm, and the blade height was 7 mm.

The flexible rotor blades were evaluated on a hydrodynamic test setup with 40% wt glycerin solution as the working fluid. The setup included a motor to drive the rotor, a flow probe, pressure transducers upstream and downstream of the rotor, and a pinch valve to vary the downstream resistance. The mounted rotor was driven at 3000 RPM, and the flow produced by the rotor was measured as the pinch valve incrementally increased downstream resistance. The study then scaled the hydrodynamic results to achieve pediatric and adult pressure/flow conditions of 70 mmHg-2 L/min and 90 mmHg-5 L/min, respectively. To analyze the off-design point operation, the study calculated the gradient of the pressure/flow curve at two operating conditions using Equations 1 and 2, respectively.

$${gradient}_{peds} = \frac{{P}_{1} - {P}_{3}}{1 - 3} mm {Hg ( L / min )}^{- 1} \qquad Eq . 1$$

$${gradient}_{adult} = \frac{{P}_{4} - {P}_{6}}{4 - 6} mm {Hg ( L / min )}^{- 1} \qquad Eq . 2$$

For a flexible rotor blade with an outlet angle of 30°, the operating speed for pediatric and adult pressure/flow conditions was 2550 RPM and 3230 RPM, respectively. The gradient at the pediatric and adult operating conditions was—6.14 mmHg/(L/min)⁻¹ and—11.12 mmHg/(L/min)⁻¹. FIG. 8B shows the results for a flexible rotor blade with 30° outlet angle.

Analysis between rigid and flexible Rotor Blades. The study evaluated the performance between flexible and rigid rotor blades. The study chose three designs for producing rigid and flexible rotor blade LVAD prototypes each. The blade thickness and height were fixed at 1.2 mm and 7 mm, respectively. The inlet angle of the blades was set at 20° whereas the outlet angle was varied from 30° to 70° with 20° increments as shown in FIG. 8C. The study 3D-printed the rigid rotor blades were 3D printed using a J850 Pro (Stratasys Ltd, MN, USA) whereas the study cast flexible rotor blades using polyurethane resin of shore hardness 60A. Results are presented and discussed in relation to FIG. 3F.

In vitro Hemocompatibility Assessment. FIG. 8D shows an evaluation on the blood damage caused by both rigid and flexible blade LVAD prototypes using porcine blood in a blood circulation loop. As shown in FIG. 8D, the test system included a motor to drive the rotor, a flow probe, pressure transducers upstream and downstream of the rotor and a pinch valve to vary the downstream resistance.

The blood circulation loop (BCL) was tuned to the adult physiological condition and blood samples were collected every 60 minutes over a period of 6 hours. The blood samples were centrifuged to prepare plasma. Hemolysis and platelet activities were quantified using commercially available enzyme-linked immunoassays (ELISA).

Hydrodynamic Performance Evaluation. The study also evaluated the hydrodynamic performance of the LVAD prototypes on a benchtop setup. The study used a blood-mimicking 40% wt glycerin solution as the working fluid. The mounted rotor was driven at 3000 RPM, and the flow produced by the rotor was measured as the pinch valve incrementally increased downstream resistance. To characterize hydrodynamic performance, the study measured the gradient of the pressure-flow curve and the efficiency using Eq. 2.

For a rigid rotor blade with an outlet angle of 30°, the gradient at adult operating conditions was—11.52 mmHg/(L/min)⁻¹, whereas its flexible counterpart had a gradient of—11.12 mmHg/(L/min)⁻¹. FIG. 8E shows that as the outlet angle increased, the pressure/flow gradient decreased for rigid rotor blade prototypes.

### SLIC Coating Evaluation

The study characterized the performance and flow of the LVAD configured with SLIC coating. The optimized flexible LVAD with or without SLIC coating are examined in the benchtop flow loop experiments. In addition to performance characterizations, the study utilized an advanced 3D time-resolved particle tracking velocimetry system to directly quantify the hemolysis index of the LVAD with/without coatings and calibrate the results obtained from CFD. Four cameras are used to record the particle images from four different viewing angles, and a state-of-the-art particle tracking algorithm, Shake-the-box, was used to track the particle trajectories as they enter and leave the pump. By integrating the shear stress experienced by individual particles along their trajectories, the HI was directly computed and compared.

Characterization of Physical and Chemical Inhomogeneities of SLIC Coating. The study characterized the SLIC coatings fabricated to assess the physical and chemical inhomogeneities and the resulting slipperiness.

Atomic force microscopy (AFM) and Electrostatic force microscopy (EFM). The study utilized a Bruker MultiMode 8-HR AFM with silicon nitride probes in the ScanAsyst mode to characterize surface roughness and then determined the physical inhomogeneities. The study scanned 4 μm×4 μm areas at a rate of 1 Hz to acquire at least 30 images. The images are analyzed with Nanoscope Analysis software to obtain the root mean square roughness Rᵣₘₛ. Additionally, with EFM accessories, the study characterized the surface charges to assess homogeneity in surface charge distribution.

X-ray photoelectron spectroscopy (XPS). The study characterized the near-surface chemical composition at multiple locations using XPS. to assess the chemical inhomogeneities. The study then characterized surface chemistry using Physical Electronics PHI- 5800 XPS. Analysis may be conducted using a monochromatic Al Kα X-ray source operated at 15 kV, and photoelectrons may be collected at a takeoff angle of 45° relative to the sample surface. XPS data may be acquired from at least 15 spatially different locations on the surface, and the spectral analysis will be conducted using PHI Multipak software.

Ellipsometry. The study assessed the chemical inhomogeneities by estimating the grafting density via thickness measurements using ellipsometry. The thickness of SLIC coatings are measured using a variable angle spectroscopic ellipsometer (VASE-VB-250). A spectral scan of the surface may be collected between 500 nm and 900 nm for an incident angle between 55° and 75° with an increment of 5°. The thickness of the PEG layer (refractive index=1.45) may be determined using a three-layer planar model (air/PEG/silica) of a solid surface from the collected spectra. Per the sample, the study conducted at least 15 measurements at different locations.

Raman spectroscopy. The study characterized the presence or absence of ice-like hydration layers on the SLIC coating with angle-resolved TIR Raman spectroscopy (Horiba XploRA PLUS) with a UV laser. The spectral scan was conducted at laser wavelengths of 266 nm and 366 nm. With a penetration depth of ~2 nm, Raman spectra may result in a detailed chemical fingerprint of the samples. The study employed a 3000-3400 cm-1 wavelength range to detect a clear peak difference between a more ordered ice-like hydration layer and a more disordered water-like hydration layer.

Contact angle goniometry and Force tensiometry: The study assessed the quasi-static slipperiness by measuring the contact angles and sliding angles of whole blood and its components using optical goniometry and dynamic slipperiness by measuring the shear forces using a force tensiometer. The study measured the contact angles and sliding angles of whole blood and its components with 20 μL sessile droplets using a contact angle goniometer/tensiometer (Rame-Hart 260) inside a chamber with controlled humidity. The study assessed the dynamic slipperiness by measuring lateral adhesion force using a force tensiometer (Kruss Force Tensiometer K 100C). Through lateral adhesion force measurements, the substrates with SLIC coatings with ice-like hydration layers may display lower lateral adhesion forces compared to substrates without SLIC coatings. The study may perform at least 10 measurements for each substrate.

SLIC Coatings Material Interactions. The study evaluated blood-material interactions of SLIC coatings fabricated both under static and dynamic conditions.

Preliminary Results. Building on the design principles of hydrophilicity and slipperiness, the study fabricated SLIC coatings on a wide variety of polymers, including rigid acrylics and flexible polyurethanes (the materials of choice for the LVADs). The study employed polyurethane substrates with polyethylene glycol (PEG; Mol. Wt. ~500 Da) brushed via TEOS modification followed by silanization.

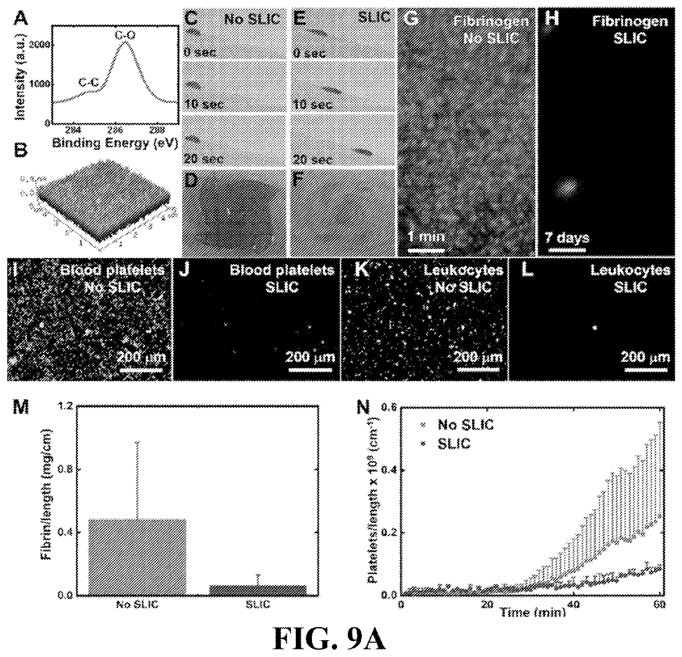

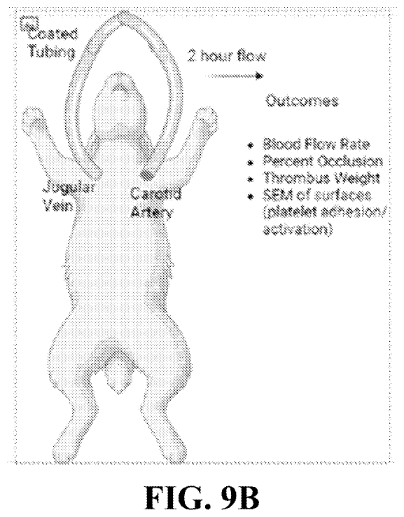

FIG. 9A shows that the PEGylation was evident from the high-resolution C1s XPS spectra. The PEGylation rendered the surface hydrophilic with a contact angle of θ=35°. Further, the surfaces displayed low chemical inhomogeneity due to the ultra-high grafting density of about 1.3 PEG brushes per nm² (based on ellipsometry measurements). While FIG. 9A, subpanel C shows that blood analog droplets cannot slide past conventional hydrophilic surfaces, FIG. 9A, subpanel E shows that they can easily slide past SLIC coatings because of the low contact angle hysteresis of Δθ<10°, which in turn arises from the low physical and chemical inhomogeneities. FIG. 9A, subpanels D and F show that LVAD surfaces with SLIC coating had minimal adhesion of blood analog. This was a macroscopic consequence of ice-like hydration layers at the molecular scale, which results in dislodging of adsorbed proteins due to slipperiness and low adhesion.

The preliminary study observed negligible fibrinogen adsorption with SLIC-coated surfaces incubated for 1 week, as shown in FIG. 9A, subpanel G and subpanel H. Further, the preliminary study also indicated virtually negligible platelet adhesion in FIGS. 9A, subpanel I and subpanel J and leukocyte adhesion in FIGS. 9A, subpanel K and subpanel L for SLIC coatings. This was in contrast to quick (~1 min) and at least an order of magnitude higher amount of fibrinogen adsorption, platelet adhesion, and leukocyte adhesion on polyurethane surfaces. The in vitro preliminary studies constitute an advance as even the state-of-the-art coatings cannot prevent protein adsorption for more than a few minutes.

In vitro analysis of thrombosis in quasi-static conditions. Several interactions occur after a material contacts blood after protein absorption. The study characterized the interactions under quasi-static conditions with and without SLIC coatings. The study obtained platelet-poor (PPP) or rich plasma (PRP) via centrifugation from human-citrated whole blood (ZenBio, NC). Whole blood may be centrifuged at 300 g for 15 mins to obtain Platelet Rich Plasma (PRP, the plasma with platelets and leukocytes, and no erythrocytes). The PRP was further centrifuged twice at 500 g for 10 mins at 35° C. to obtain Platelet Poor Plasma (PPP, the plasma without platelets, leukocytes, and erythrocytes). The study utilized the resulting PPP in protein binding, which occurs when blood comes in contact with a surface. The study used PRP to evaluate the later stages of blood-material interactions. All the biological experiments were conducted on at least 5 surfaces with at least 3 different cell populations from different donors (nmin=15).

Fibrinogen and albumin binding from PPP on different surfaces. Thrombin, a key protein responsible for platelet activation, initiated the clotting cascade. The enzyme thrombin transforms fibrinogen into fibrin monomers, which, under normal conditions, may form polymeric fibrin fibers to form a clot (thrombus). So, fibrinogen was related to blood clotting, and preventing its adsorption may prevent clot formation. To evaluate the pro-coagulant activity, the study evaluated fibrinogen binding from PPP on different surfaces using a human fibrinogen ELISA assay. Albumin also acts as a carrier for (1) antithrombin, which keeps the clotting enzyme thrombin from working unless needed, and (2) heparin cofactor, which was necessary for the anticlotting action of heparin. Quartz crystal microbalance (QCM) was used to quantitatively evaluate total protein adsorption on the surfaces from PPP. QCM was a sensitive technique that measures nanograms of proteins adsorbed on the surface based on the change in the oscillation frequency of Quartz. Additionally, protein adsorption from PPP on different surfaces was measured using quantitative XPS by determining the contribution of N—C═O (amide) peak, which is characteristic of the proteins adsorbed on the surface.

Adhesion forces of proteins by atomic force microscopy. The adhesive force between the protein and the surface was a measure of how strongly the protein was adsorbed or bound to the surface. To estimate how strongly the proteins are adsorbed or bound to the surface, the study estimated the adhesive forces using atomic force microscopy (AFM). Silicon nitride AFM tips were adsorbed with known concentrations (e.g., 10 mg/ml) of fibrinogen and albumin solutions. Then, each modified AFM tip with fibrinogen or albumin may be brought in contact (i.e., engaged) with and then detached (i.e., disengaged) from different surfaces, and simultaneously, the force required to detach from the surface may be measured by immersing the surfaces in PBS. The study then imaged protein morphology in tapping mode with PBS and air for comparison. The study made measurements at 30 random locations on each surface.

Platelet adhesion, activation, and platelet-leukocyte complex formation. The study stained the cells with calcein-AM live stain to evaluate the platelet adhesion on different surfaces from PRP. The study utilized an LDH assay to characterize platelet viability. Platelet activation was evaluated via SEM. Platelet and leukocyte activation were investigated by immunofluorescence staining and western blotting for specific marker proteins, P-selectin (for platelets), and CD-45 (for leukocytes). P-selectin expression by platelets can play a role in the initial recruitment of leukocytes to the site of inflammation, leading to platelet aggregation. CD-45 is a transmembrane protein present in human leukocytes that plays a role in signal transduction.

Contact and complement activation. Contact activation can impede the hemocompatibility of blood-contacting materials. Proteins involved in the contact activation system (e.g., factors VII, IX, prekallikrein, etc.) are profibrinolytic, anti-adhesive, procoagulant, and pro-inflammatory. The study evaluated the activity of the kallikrein-a2-macroglobulin complex using an antigen assay to determine the degree of contact activation since all kallikrein was released from the surface of the material due to exposure to PRP.

Thrombin generation from PRP. Thrombin is an enzyme of the coagulation cascade whose measurement gives direct information about the thrombogenicity of a material (i.e., its ability to form blood clots). In normal plasma, thrombin may be captured into the fibrin meshwork and is rapidly inactivated by antithrombin III or other antiproteases. The short half-life of thrombin can hamper its accurate enzymatic determination. The study assayed the surface-exposed PRP to determine the thrombin activity using the thrombin generation assay.

Whole blood clotting and hemolytic activity. To evaluate whole blood clotting, the study dropped 5 μl of the blood on the different surfaces. The blood was allowed to clot for up to 60 mins, and the free hemoglobin concentration in the unclotted blood was measured at 15 mins intervals. The value of absorbance was directly proportional to the concentration of free hemoglobin in DI water and was an indirect measurement of the surface's ability to promote clotting. The study investigated the hemolytic activity of different surfaces using a material hemolytic assay.

In vitro analysis of thrombosis under dynamic flow. The study evaluated the ability of SLIC coatings to inhibit clot formation in real time under physiologically relevant flow conditions using a custom fluidics-based microscopy approach. A glass slide of antithrombotic coatings and a PDMS device was assembled over the slide. The device was then mounted on a widefield microscope with an automated stage for imaging, and whole blood, plasma, or isolated clotting components (i.e., fibrinogen and thrombin) may be flowed into the device. The study included fluorescently labeled fibrinogen to allow monitoring of clot formation/growth. The study took images at 5 min intervals for 12 hrs to monitor clot formation, growth, and flow rates, at physiologically relevant shear rates of 1, 10, 100, and 1000 s⁻¹.

Ex vivo analysis of thrombosis in rabbit model. Through an iterative approach between fabrication and characterization of SLIC coatings and in vitro analysis of thrombosis, the study identified and evaluated the two promising SLIC coatings in a rabbit model of extracorporeal circulation. The study tested three experimental groups using rigid acrylic and flexible polyurethane tubings (depending on results from in vitro studies), with and without the two SLIC coatings. The SLIC coatings may coat the entire blood-contacting area of the tubings. The study utilized New Zealand white rabbits (2.5-3.5 kg), with groups split evenly between male and female rabbits. After induction of anesthesia, the left carotid artery and right jugular vein were isolated, and an arterial/venous extracorporeal circuit may be connected by cannulating the left carotid artery and the right external jugular vein as shown in FIG. 9B. Blood flow through the shunt was initiated by unclamping the arterial and venous sides of the extracorporeal circuit and then blood flow was monitored for 2 hours, at which point the tubing was removed. The study photographed cross-sections of the tubing to determine percent occlusion of the circuits. The study collected, weighed, prepared clots in the tubing for SEM analysis.

Blood Damage Performance Characterization. The study used in vitro experiments to evaluate the performance and blood damage potential of the flexible rotors. Rotor blades with hardness ranging from Shore 10A to 100A were studied. Flexible water-clear polyurethanes (BJB Enterprise, Tustin, CA) were used for the rotor to view the rotor passage. The acrylic casing was machined and coated with a layer of the same polyurethane. Acrylics are low-cost materials that have hemocompatibility and may be made by additive manufacturing in the future to lower the cost further. A shaft-mount torque meter was used to monitor the input power, and the efficiency was calculated by using the pressure and flow data. All the flexible rotors were tested under 2000-6000 RPMs and 2-8 L/min to determine their performances, and results were compared with those obtained from a rigid rotor. The study focused on the performance and efficiency of the flexible rotors under off-design conditions and compared them with their rigid counterpart.

Durability Test of Flexible Rotor. Since the blade is flexible, durability is a concern. The study used a custom-built accelerated hydrodynamic testing rig. Briefly, a rotor was kept operating at 1.5 times the normal speed (>4500 RPM) and under two times the normal pressure rise (>180 mmHg). The performance was monitored continuously by a data acquisition system, and the time to fail was recorded.

Performance under pulsatile inflow conditions. The study tested the flexible LVAD with stented inlet and SLIC coatings under pulsatile inflow conditions. The study deployed and anchored the model to the downstream of a left-heart simulator. In the simulator, a bladder pump is driven by compressed air and controlled by solenoid valves to generate pulsatile flows. By adjusting the resistor and compliance chamber downstream of the pump, physiological and pathological waveforms were obtained. The study used flow and pressure waveforms mimicking those from end-stage heart failure patients. While the inlet of the LVAD is subjected to this pulsatile inflow condition, the pressure rise and efficiency of the pump were recorded at 3000 RPM. The averaged efficiency over at least 100 cardiac cycles may be used to determine the efficacy of the flexible rotors.

Flow field measurements and direct blood damage quantification. The study used a selection of ten flexible rotors that have the best averaged efficiency. The study used high-resolution 2D PIV to visualize the flow in the rotor passage and measure the shear rate in regions of interest. Briefly, the flow was seeded with tiny particles coated with a fluorescent dye. To facilitate visualization, blood-mimicking fluid (Water-glycerin-NaI) that has the same refractive index of the acrylic casing (n=1.49) and the same viscosity of blood may be used. The laser beam emitted from a high-speed Nd:YLF laser was converted by a series of optics into a thin sheet (<1 mm) that illuminates the flow. A high-speed camera placed perpendicular to the laser sheet recorded the fluorescent particle image through a band-pass filter, which blocks all other unwanted reflections. The high-resolution flow field was calculated by a cross-correlation-based code package, DaVis 10 (LaVision GmbH). The study acquired measurements in a series of axial planes covering from the inlet to the outlet of the LVAD model. For each plane and rotor blade orientation (phase), 1000 instantaneous visualizations were captured to ensure convergence for statistical quantities such as turbulent kinetic energy and Reynolds stresses. They were used to calibrate the turbulence models in CFD. The averaged shear rate was directly calculated by applying the sum-of-correlation algorithm described by Westerweel et al. The resolution of the measurements can reach up to a single pixel size of the camera (~10 μm), more than enough for accurate shear rate quantification, even in the rotor tip gap.

Characterization of Anti-thrombotic Response. The study characterized the thrombotic response of all the flexible LVADs with SLIC coatings fabricated under quasi-static and dynamic conditions. The study used a minimum sample size of n=10 in each test and compared the results from a rigid rotor with or without SLIC coatings.

Quasi-steady conditions. For the quasi-steady condition, whole human blood was centrifuged to separate plasma from the red blood cells. The flexible rotors were incubated in plasma. Fibrinogen, albumin, and immunoglobulin-G adsorption on LVAD materials were evaluated using an ELISA to understand how serum proteins interact with the surfaces, leading to assessing the sample's clotting properties and their interaction with whole blood. Whole human blood was placed on the samples and allowed to clot, and free hemoglobin concentration may be measured. The study imaged the surfaces via SEM to visualize the fibrin clot formation.

For the dynamic condition, about 150 mL of whole human blood were anticoagulated with 3.2% sodium citrate (blood to citrate volumetric ratio=10:1), introduced into the flow loop, and recalcified (citrate to Ca2+ molar ratio=8:1). Pressure transducers upstream and downstream of the test LVAD measured the pressures. The pump operated at a constant RPM but under varying resistance conditions of the loop to mimic the complex inflow in an actual patient. The resistance of the loop was regulated by a pneumatic pinch valve placed around the tubing upstream of the pump, and a sinusoidal waveform may be used to control the valve. The study used D-Dimer (a fibrin degradation product) and thrombin-antithrombin complex (TAT, a protein complex of thrombin and antithrombin) to quantify thrombogenicity. The study measured concentration using commercially available ELISA kits. The levels of plasma-free hemoglobin (pfHgb) and plasma lactate dehydrogenase (LDH) were used to evaluate hemolysis.

Dynamic conditions. The study also conducted the characterization of the thrombotic response above under dynamic conditions. Briefly, about 150 mL of whole human blood may be anticoagulated with 3.2% sodium citrate (blood to citrate volumetric ratio=10:1), introduced into the flow loop, and recalcified (citrate to Ca2+ molar ratio=8:1). The study used a linear actuator to drive blood flow physiologically. The piston was housed inside a titanium tube connected downstream to an appropriate test chamber containing the LVAD. A bypass tube facilitates the return flow of blood to the titanium tube during diastole. Pressure transducers upstream and downstream of the test valve measure the pressures. A latex bag housed inside an acrylic chamber (downstream of the leaflet) serves as the compliance chamber. The study indicated that the pulsatile blood loop may capture the early biochemical changes relevant to blood clotting.

### Design and Test of Wireless Energy Transfer and Communication System

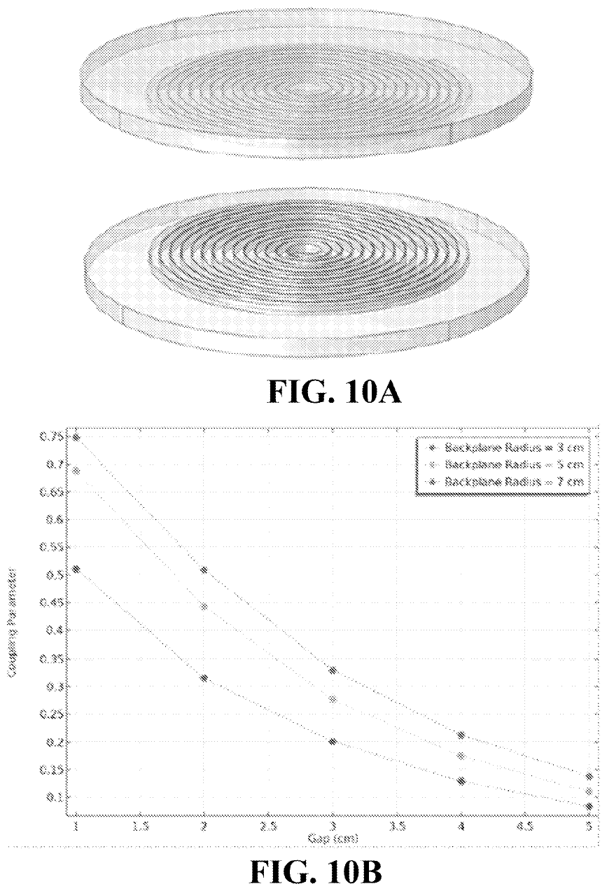

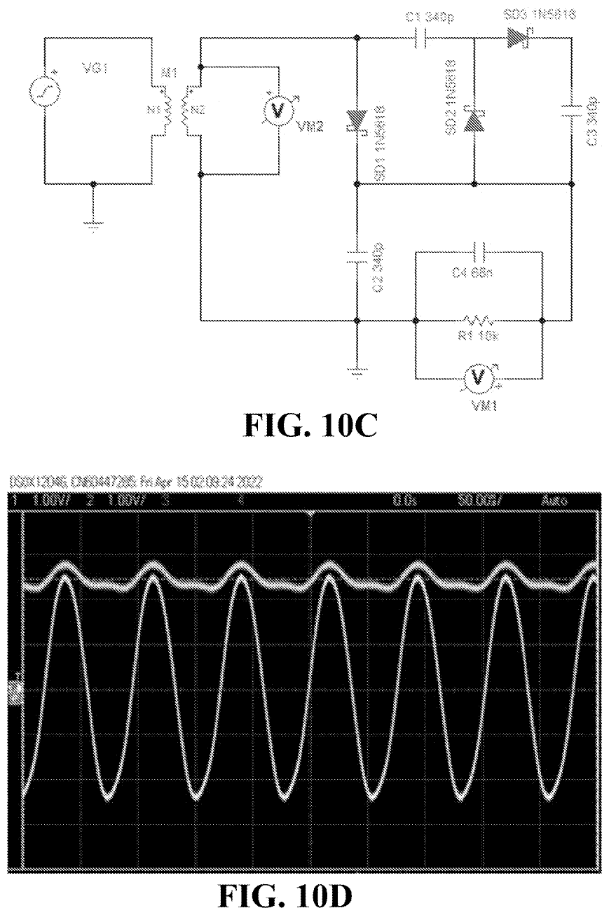

The study evaluated the design, placement, and control of the antennas for the energy transfer system. FIG. 10A shows a COMSOL 6.0 coil model with backplane layers for improved coupling factor. The improved design of internal and external antennas and backplanes may increase energy efficiency and reduce heating losses.

Finite element analysis was used to design the flexible antennas to follow body contours to reduce losses due to external to internal gap variations. In a preliminary study, finite element analysis of electromagnetic fields using COMSOL 6.0, SPICE circuit analysis (FIG. 10C) of the rectifier design, and validation through physical testing was done. Each simulation was run at different gaps between the two antennas to represent varying skin and tissue thicknesses among the population. The geometry of the antenna was measured and modeled using COMSOL. The antennas were set parallel to each other at gap distances of 1 cm to 5 cm from each other at 1 cm increments. To take advantage of the axial symmetry and speed up computation times, the axial symmetric geometry was used, as shown in FIG. 10A.

A Field Programmable Gate Array (FPGA) programmable hardware board with a variable gain power amplifier (PA) was used to generate the input signal to the transmit antenna (FIG. 10D).

The study used two of the same antennas with the same geometry and inductance values. Three characteristics were extracted from the results at each gap size. The inductance of the antenna (L), the mutual inductance between the two antennas (M), and the coupling parameter (k). The k parameter is a ratio between the mutual inductance between the antenna and the inductance of the antenna. The closer the k parameter is to 1, the more efficient the power transfer between the antennas.

Wireless transmission utilizes non flexible materials for their designs. Flexible antennas may allow for better form fitting to the human body, increasing comfort. The effects of curvature on the coupling between the antenna may be modeled using COMSOL 2D geometry. The curvature of the antenna may be done following the arc of a circle while keeping the length of the antenna the same. To change the curvature, the radius of the circle may be adjusted. The smaller the radius, the larger the degree of curvature.

FIG. 10B shows the results from the finite element analysis simulations, which show that the gap distance between the two antennas increases as the coupling factor decreases. This means that the efficiency of power transfer from the transmitter to the receiver may also decrease. The study developed custom flexible antennas with appropriate backplane material to improve efficiency and to be shaped to a patient's body. The study determined the size, shape, and number of coil turns for improved energy transfer.

The study collected data from several animal studies, including (i) conduction velocity (the velocity at which a depolarization wave moves through the myocardium. This may aid in identification of areas of slow conduction that require correction), (ii) myocardial voltage (the voltage of the myocardium may aid in identification of the extent of injury or scarring in a quantifiable way), (iii) inducibility threshold (the lowest level of either pacing or medication to create a sustained arrhythmia), and (iv) depolarization and repolarization heterogeneities: differences in conduction between various cardiac regions, helping to identify areas of injury and scar.

The study performed the data collection with a standard electrophysiology study utilizing high resolution mapping catheters (HD Grid, Abbott, IL) with an accompanying electrophysiologic mapping study system (Ensite, Abbott, IL). The electroanatomical mapping provides high-resolution data to assess the electrical conduction of the myocardium around the LVAD and the other chambers of the heart, with the LV and the LA being of the most interest.

The study performed electroanatomical mapping before and after LVAD implantation and at the end of the longer chronic implant time point to establish a baseline for comparison, assess acute changes, and assess longer-term outcomes in changes to the native conduction patterns. Additionally, a 12-lead surface ECG may be utilized for global conduction assessment for the duration of all animal studies. Ultimately, the 12-lead data may be used as a surrogate for mapping data in a future iteration of the LVAD, in which the LVAD may use sensed 12-lead data to optimize pump settings to improve cardiac health.

### Evaluation of Heartbeat Synchronized Speed Modulation of a Continuous Flow LVAD Using a Mock Circulatory Loop

The study evaluated heart beat synchronized speed modulation. Current continuous flow LVADs (CF-LVAD) create flow that is counter to the natural pulsatile flow of the heart and vascular system, leading to decreased aortic blood pressure, which is linked to increased internal bleeding. The study employed sensor input to modulate the speed of a CF-LVAD in synchrony with the heart rhythm. The study also evaluated, using a Frank-Starling controlled mock circulatory loop (MCL), the hemodynamic response of speed modulation.

The study used a 3D-printed centrifugal CF-LVAD driven by a brushless DC motor. The study created an algorithm to control the CF-LVAD speed using left ventricle pressure sensor input to time the speed changes. The CF-LVAD controller and the MCL controller ran independently from each other. The CF-LVAD inlet and outlet were attached to the MCL's left ventricle and aorta, respectively. The MCL heartrate was set to a severe heart failure condition with a heart rate of 60 BPM.

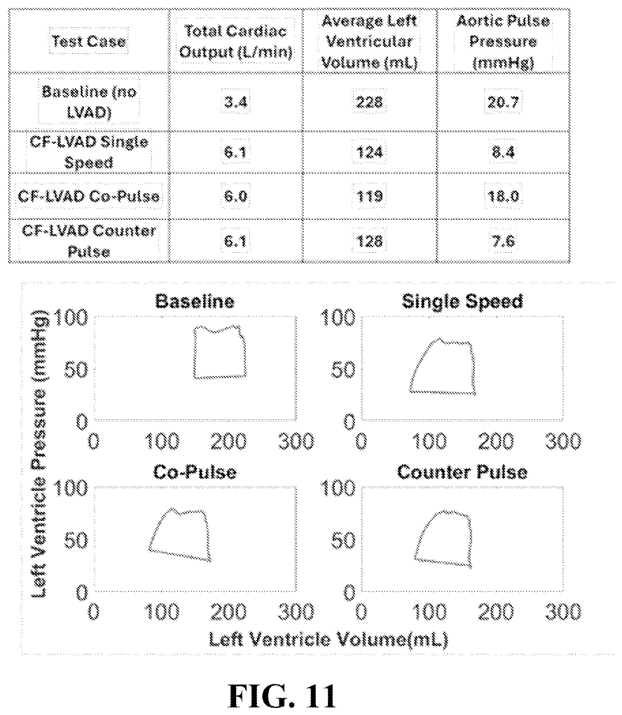

FIG. 11 shows three timing test cases in addition to a baseline with no LVAD. In single speed flow, the pump ran at a set speed of 2200 RPM during the entire cardiac cycle. The speed was set to reach a cardiac output of 6 L/min. In co-pulse, the LVAD reached a peak speed of 2600 RPM during systole and a minimum speed of 1900 RPM during diastole. In counter pulse, the LVAD reached a minimum speed of 2000 RPM during systole and a peak speed of 2500 RPM during diastole. The minimum speeds in the speed modulation cases were set to prevent backflow through the CF-LVAD, and the peak speeds were set to reach an average cardiac output of 6 L/min.

To evaluate the performance, the study ran the MCL at the baseline case and the three CF-LVAD speed timing cases for two minutes each. The study used the MCL sensors to measure and record the aortic pressure, total cardiac output and left ventricle volume and pressure.

As shown in FIG. 11, all three CF-LVAD cases were able to increase the total cardiac output while unloading the heart. By using co-pulse speed modulation on a CF-LVAD, the aortic pulse pressure was over double the aortic pulse pressure of the two other CF-LVAD cases and reached 87% of the baseline case aortic pulse pressure. The co-pulse speed modulation achieved increased cardiac output, unloaded the left ventricle, and, in addition, maintained pulsatile flow and pulse pressure closer to the physiological norm.

The study evaluated control algorithms for improved dynamic LVAD performance, including an electrophysiology investigation to provide data on the effects of an LVAD on electrical conduction patterns pre and post-implantation. The data analysis can be used to improve control of LVAD pump speed in various dynamic situations and lead to better long-term outcomes.

Numerical Modeling of BLDC Motor. The study modeled a BLDC motor in a FEM software package (Comsol 5.0). The outer diameter of the BLDC stator was constrained to 35 mm. The study evaluated different gap sizes between the rotor and stator to analyze the motor torque as well as the force generated with radial and axial displacement. The diameters of the stator the rotor are optimized to ensure that no flux saturation may occur at the motor's operational speed of 3,000 rpm. The study optimized the length of the BLDC motor to ensure that adequate motor torque may be generated to meet the hydraulic torque requirement (a hydraulic torque of 10 mNm was generated based on the hydraulic tests based on the test data). The FEM analysis provides an induced voltage, which may then be used to calculate the back electromotive force (EMF) constant kE to estimate the motor torque during operation. The study then calculated the torque constant. The air gap magnetic field Bg is also recorded for the analytical calculation of the magnetic force generated due to rotor eccentricity. The study then analyzed the magnetic force present with incremental axial and radial rotor displacement to calculate the radial and axial stiffness of the BLDC motor.

Design and fabrication of the Magnetic Bearing System (MBS). The rotation controlled by the motor was defined as θz, translation along the rotational axis was z, radial translations are x and y, and rotation (inclination) of each axe are θₓ, θ_{y}. The BLDC motor controls rotation, and maglev technology was chosen to control the other five degrees of freedom. Radial passive magnets were fixed to the rotatory impeller, and three copper-wired permanent magnets were attached near the inlet cannula, opposite to the stator. The study radially magnetized all permanent magnets for the passive magnetic bearing, so the repulsive force between the rotor and stator permanent magnets stabilizes the radial direction (x, y) and the inclination (θₓ, θ_{y}) passively. Due to Earnshaw's theorem, a magnetic system that was passively stable in the radial direction may be unstable in the axial direction. If the axial position was stabilized by an active magnetic bearing featuring reluctance forces, the passive stiffness should be calculated for the rotor dynamic model. Furthermore, these stiffness values are dependent on the coil current, and the negative radial stiffness of the motor should be included in the design. The design goal of the passive magnetic bearing was that the rotor and the stator do not contact each other even if the rotor was subjected to more than 5G acceleration. With a rotary impeller weight of 5 g (0.005 kg) and rotor magnets weight of 50 g (0.05 kg), the force required to generate axial magnetic bearings under acceleration of 5 G was 0.055 kg×9.8 m/s2×5=27 N. In summary, all 6 degrees of freedom are stabilized: θz was rotated by the BLDC, x, y, θₓ, and θ_{y} are passively stabilized, and z was actively stabilized.

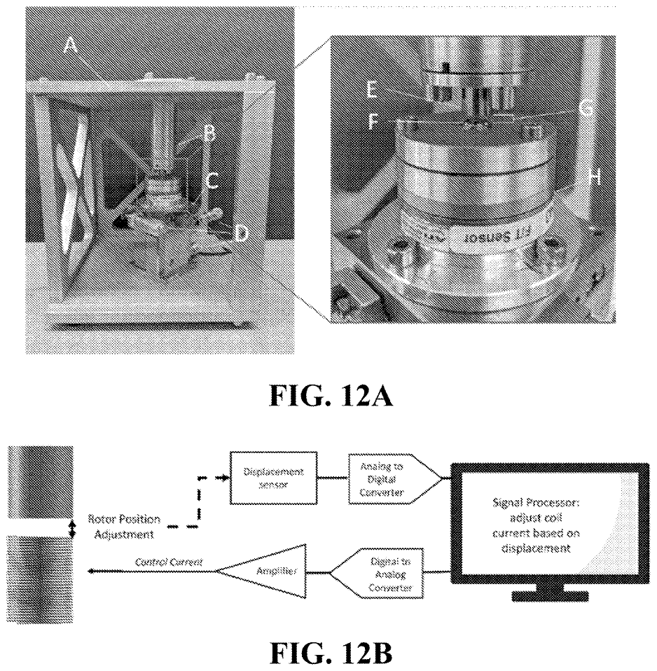

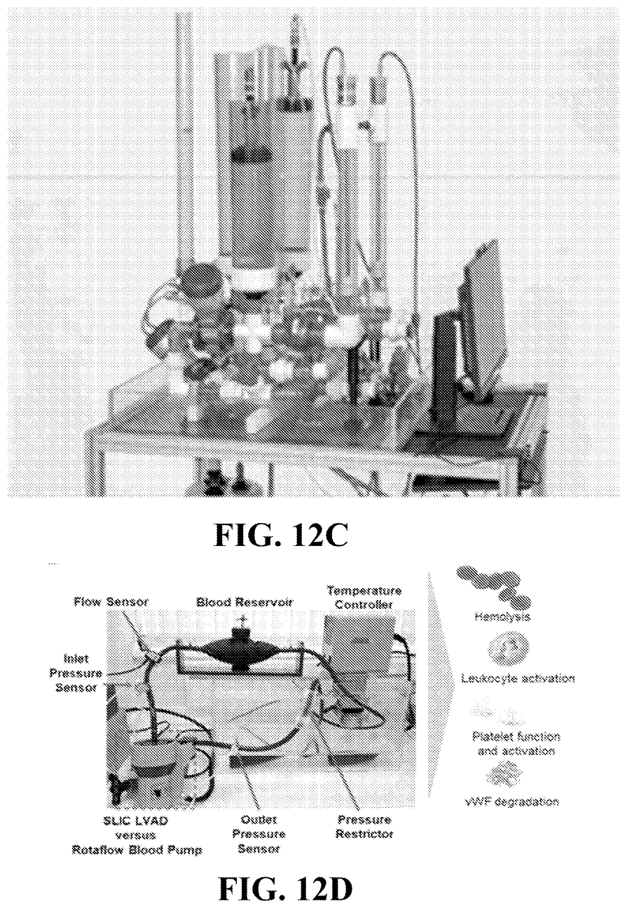

FIG. 12A shows an outline of the test bench for the motor force measurement. To obtain precise force measurements with high spatial fidelity, a force transducer (ATI Industrial Automation, Apex, NC, USA) with a 1/512 N resolution was mounted on a micrometer-driven x-y-z stage (MiSUMi Group, Inc., Tokyo, Japan).

The stator section was fixed to the force transducer. The rotor was set to a highly accurate x-y-z stage moved by a micrometer head. The displacement of the rotor was measured with a laser displacement sensor with higher precision than the micrometer. With this experiment, it was possible to measure the radial stiffness and the negative axial stiffness accurately. Several different permanent magnet designs are tested with varied thicknesses and diameters for the rotor; all are made of rare earth magnets (NeFeB). The permanent magnets are measured for the static performance. Accurately measuring the rigidity of the permanent magnets in the radial and axial directions was extremely important for the design and control of axial magnetic bearings.

The maximum axial displacement of the rotor should be ±0.2 mm, and the repulsive force in the axial direction was the sum of the electro-magnetic force between the motor stator and rotor and the rotor force due to gravity. The axial active magnetic bearing should balance this force to achieve stable non-contact rotor suspension. To sustain patient quality of life, the rotor should remain stably suspended during day-to-day human activities, and the maglev system should operate on 5 W or less to improve battery life. FEM magnetic field analysis enables precise examination, including stator shape, stator material, number of coil windings, winding wire diameter, and size of the permanent magnet. Based on the simulation results, the study designed the entire maglev motor, including the displacement sensing system, using a 3D CAD system.

Stabilizing Control and Performance Evaluation of the Maglev Motor System. The study implemented the maglev and rotation controller. FIG. 12B shows the diagram of the magnetically levitated motor control system. The control system consists of the maglev motor, the displacement sensor, the analog/digital converter (ADC), the controller, the digital/analog converter (DAC), and the power amplifier.

Position feedback are provided by a sensor to control the axial direction actively. The displacement signal was fed into the controller via ADC. A high-speed digital signal processor computes the maglev electrical current to reposition the rotor and feeds that value back to the current amplifier via the DAC. Then, the amplifier provides the specified control current back to the maglev coil to dynamically produce a tuned suspension force. The study developed the controller in the MATLAB/Simulink environment and implemented it on a dSPACE controller board. The study measured the static and dynamic characteristics of the maglev motor system in response to an array of perturbations, including both rigid body (patient motion) and fluid (hemodynamic) stimuli.

Experimental Testing of the Motor Drive System. Based on the numerical results, the study developed and manufactured the motor rotor and stator separately. The motor was driven by a pulse width modulation (PWM) amplifier (Koford Inc., S24V10A-H3) and the power source (TKD-Lambda Inc., CME350A-24). The PWM amplifier specifies the motor target speed by the reference input voltage from 0 V to 5 V as motor speed ranges from 0 min⁻¹ to about 26,000 min⁻¹. The PWM amplifier fed the control current to the BLDC motor via the terminal box. The terminal box connected the power meter (Yokogawa Inc., WT-1800), dynamic motor torque test rig, BLDC motor, and PWM amplifier. The motor mounting jig fixed the motor to the base with the motor coupling connected to the motor and the torque meter (Sugawara laboratory, TB-200NM). The controller (Sugawara laboratory, DMC-2) controlled the torque meter, which specifies the automatic measurement sequence. The study may evaluate the motor torque and efficiency.

Design characterization and validation. To examine levitation characteristics, the study measured the impulse responses in 40 wt % glycerol at 24° C. with a rotational speed of 3,000 rpm. An external electromagnet created an impulse disturbance to the levitated rotor via the pump casing. The response of the controller was evaluated by measuring the time until the vibration amplitude converges to 5% or less of the maximum variation after the disturbance was applied. Because the axial suspension was controlled actively, the responsiveness in the axial direction may be tuned until it was fast enough. In the radial direction, the settling time may be long since passive permanent magnet bearings providing radial support do not produce a damping force. However, the damping effects of the glycerol solution should improve the stability in the radial direction.

To examine levitated rotational features, the study measured vibration amplitude in a glycerol solution environment while the rotating speed varied. The eddy current sensor measures axial displacement, and additional laser displacement sensors measure radial position and tilt movement. The study increased the rotating speed stepwise up to 5,000 rpm. After the rotor reaches a steady state speed, the study recorded the vibration amplitude of the rotor movement. The axial displacement, radial displacement, and tilt amplitude become large at each resonance frequency. However, the fluid viscosity may attenuate the rotor vibration. In terms of the stability of the maglev control, a viscous liquid was desirable. The study tuned the maglev control gains in a static state. However, when the rotor levitates and rotates, the vibration of a frequency synchronized with the rotating speed occurs, which may cause control instability. Therefore, it may be necessary to apply a tracking filter or adaptive control to reduce the vibration synchronized with the rotating speed.

System Evaluation of the SLIC LVAD on the Mock Circulatory Loop. Mock circulatory loops (MCLs) enable hemodynamic evaluation in vitro [58′], [59′] and have been used to evaluate both total artificial hearts [60′] and LVADs implanted in various locations [61′], [62′].

FIG. 12C shows that the pulsing test of the MCL provides dynamic fluid perturbations emulating what the pump may experience due to hemodynamic effects. The MCL has characteristic resistance, arterial compliance, peripheral resistance, an inertial component, and venous compliance. The heart has 4 chambers whose performance is characterized by their volumes and contractility. The volume of each ventricle may be calculated in real-time by using magnetostrictive level sensors. Combining volume with the measured pressure enables one to determine the instantaneous pressure-volume relationship throughout the cardiac cycle. The left and right ventricles are made to contract by injecting compressed air into the chambers. The contractility was controlled by electro-pneumatic air pressure regulators (ITV2010-21N2S4, SMC Corporation, Tokyo, Japan). To obtain clinically relevant results, the MCL should be able to replicate the Frank-Starling mechanism. Passive filling augments the functionality of the Frank-Starling mechanism. Because the chamber fills passively, the end-diastolic volume (EDV) depends on the state of the rest of the system. The contractility was calculated by a proportional controller dependent on the measurement of the EDV, updated each cardiac cycle.

The pump was mounted on a voice coil shaker system (MB Dynamics, OH, USA) and shaken each radially and axially during operation to create the effects of patient motion. Each perturbation was tested at multiple frequencies to identify potential resonant frequencies or flutter instabilities. As a metric of success, the impeller rotation speed and power consumption was monitored; deviations from the target will indicate that the levitation system failed to maintain system stability. This experiment provided a comprehensive evaluation of the MBS's performance in the device.

Hemocompatibility Evaluation of the Composite SLIC LVAD Device in vitro. FIG. 12D shows an in vitro blood circulatory loop for hemocompatibility tests. Hemocompatibility tests were performed in the circulatory loop with steady-state flow in accordance with ASTM standards. The study utilized a total blood volume of 450±50 mL in each test and utilized a temperature-controlled water bath to ensure that blood temperature was maintained at 37±1° C. Blood flow and pressure difference between the pump was maintained at 5.0±0.5 L/min and 100±5 mmHg, respectively. The SLIC LVAD pumped citrated blood for 6 hours, and blood samples were collected in every 60 minutes of interval time. Blood pressure, temperature, flow, rotor speed, and power consumption were recorded throughout the experiments. The study performed hemocompatibility testing of SLIC LVAD. The study also performed hemocompatibility testing using a Rotaflow blood pump as a control pump to compare to the SLIC LVAD device. Additionally, the remaining blood was kept in a blood bag and fully submerged in the water bath as a static control.

The study collected and centrifuged the loop blood samples, and the study transferred the plasma supernatant from each aliquot into 3 cuvettes and diluted with 0.1% Na₂CO₃ solution. The absorbance may be measured at 3 wavelengths (380, 415, and 450 nm), and from these, the plasma-free hemoglobin and normalized index of hemolysis (NIH) may be calculated. The study utilized flow cytometry for a quantitative measurement of activated platelets and leukocytes. The study also stained whole blood samples with CAPP2A, CD42b, and CD62P antibodies to quantify platelet activation and with CD45, CD11, CD14 antibodies and 7AAD dye solution to quantify leukocyte activation. In addition, platelet function was evaluated by an aggregometry using standard agonists of collagen, ristocetin, ADP, and TRAP-6. For vWF multimer analysis, plasma protein size was separated using agarose gel for electrophoresis and may then be transferred to a polyvinylidene difluoride membrane using the capillary blotting technique. The membrane may be incubated with anti-human vWF antibody and finally visualized and quantified using densitometry. For data analysis, the study calculated averages and standard deviations for each hemolysis, platelet activation, and function, leukocyte activation, and vWF multimer degradation. An analysis of variance may be applied to determine significant differences between tested geometries compared to differences in the bovine blood used for each test.

The SLIC LVAD device can introduce a nominal level of blood trauma due to surface roughness or a stagnation point in the system. Therefore, blood was first driven by a Rotaflow blood pump through the SLIC LVAD device without a rotor in place so that a blood trauma baseline was established. The Rotaflow blood pump has a well-documented hemocompatibility profile, allowing separation of the SLIC LVAD device blood damage and that generated by the Rotaflow blood pump. Due to the amount of blood volume needed for each experiment (450 mL) and concerns regarding human blood-borne pathogens, a bovine model was chosen.

In vivo studies of the SLIC LVAD in a Large Animal Model. To evaluate the hemocompatibility of the partial cardiac support (SLIC LVAD), an in vivo, the study can employ a large-animal model. Fifteen calves (10 for acute, 5 for 30-day long-term) may undergo an open implantation (one at a time to allow for prototype iterations between studies) of the SLIC LVAD to assess hemodynamic performance. The studies chose calves as an animal model as they are (i) more similar in size to humans, (ii) share similarities in hemodynamic physiology and microcirculatory function, and (iii) have been widely used in biomedical research to model other human conditions. Clinical (human) investigations do not allow systematic investigation of organs without the confounding effects of previous illnesses, varying degrees of organ failure, concurrent medications and illnesses, age, etc.

The study can utilize calves between 4 and 8 months old with a body weight between 75 and 100 kg. Calves may undergo intravenous anesthesia, intubation, and mechanical ventilation according to the standard procedures. The study can place the venous and arterial lines for blood sampling and pressure monitoring in the jugular vein and mammary artery, respectively. After probe and catheter placements, the study can place the calve on cardiopulmonary bypass for implantation of the SLIC LVAD. The SLIC LVAD may be implanted via thoracotomy and will be positioned transapically in the left ventricle. The outflow graft may be anastomosed to the descending aorta. The SLIC LVAD may be connected and desired, and the lungs may be re-perfused slowly, and ventilation recommenced. Once the animal are successfully weaned off the bypass, the native heart can support the circulation, with offloading of workload achieved by the SLIC LVAD. Flow probes may be placed on the pulmonary vein, ascending aorta, and outflow graft. Pressure lines may be placed in the left atrium and ascending aorta for continuous monitoring. The chest may then be closed to maintain normal physiology and thoracic pressures. The calve may remain anesthetized during a series of tests to thoroughly interrogate pump and host interactions for a maximum period of 24 hours. The study can collect serial blood samples for estimation of arterial blood gases and perform hemocompatibility testing to evaluate hemolysis, platelet activation, leukocyte activation, and vWF degradation and its activity. The hemodynamic performance of the SLIC LVAD may be measured at different pump speeds. The rotational speed, power consumption from the BLDC motor, power consumption, pump temperature, and pump graft flow may be continuously recorded. Additionally, native cardiac performance may be manipulated with vasoactive and inotropic agents to assess the SLIC LVAD's performance under different physiological conditions. Phenylephrine (2 ug/kg) bolus may be given to assess the pump's performance during a high systemic vascular resistance. The study may use dobutamine infusion (10 to 40 ug/kg/min) to create a hyperdynamic state. The study assessed each hemodynamic change for up to 2 hours, and the study may give time between each physiological condition manipulation to allow for a steady state to be regained.

In the 30-day non-GLP study, the animal would be recovered after implantation. Throughout the postoperative course, the calf may receive medication to prevent pain and antibiotic therapy to maintain and balance gut microbial organisms and to prevent the formation of gastric ulcers. The study may collect data postoperatively and include measurements of pump variables such as speed, power, and flow through the outflow graft. The study collected biometric data to monitor the calf's condition, including heart rate, body temperature, arterial/venous blood gases, hematocrit and total protein levels, activated clotting times, and AoP. To monitor for blood trauma or organ damage throughout the 30-day period, blood samples may be drawn accordingly, including standard hematologic testing, plasma-free hemoglobin (PFHb) levels, and coagulation and chemistry testing. A

### Machine-Learning Optimized LVAD

Determining the suitability of a patient and the proper timing for intervention can be important for successful LVAD implantation and patient recovery. Deep Learning tools that c can help assess the severity of the heart's condition in a patient may aid the specialist in making the decision. The study utilized the electrocardiogram (ECG) signals, fundamental tools in cardi-ology, as input data for the system.

Deep Learning for ECG classification has been used in recent years, with convolutional-related architectures being widely used for this end, and the potential and need for interpretability and uncertainty awareness for real-world application is evidenced. The study focused on the candidacy assessment problem and proposed ECG criteria to train the models. The study combined three models to get a report that highlights information that is useful for the physician. It also implemented interpretability and uncertainty estimation to increase the trust and applicability of the system.

ECG diagnosis criteria. For identification of the ECG diagnoses, the study discriminated the problems as being related to the left ventricle while at the same time looking for right heart problems (up to 53% of LVAD patients have right heart failure after implantation) to select LVAD candidates for identification of the ECG diagnoses. The study divided the ECG diagnosis criteria into three groups. Major criteria were Left Bundle Branch Block (LBBB), Premature Ventricular Contraction (PVC), Left Ventricular Hypertrophy (LVH), Anterior Myocardial Infarction (AMI), and Congestive Heart Failure (CHF), as well as QRS duration, which had a risk/relevance for candidacy that increases linearly starting at a duration of 110 milliseconds. Minor criteria were Inferior Myocardial Infarction (IMI) and Atrioventricular Block (AVB); and potential contraindications related to problems with the right chambers of the heart, and comprised Right Ventricular Hypertrophy (RVH), Right Bundle Branch Block (RBBB) and Right Atrial Enlargement/Overload (RAE). The output was the estimated probability of having each one of these diagnoses. The study did not use a single numerical score as a final measure of candidacy but a more informative report.

System for LVAD Candidate Assessment from ECG. The ML system included several parts: i) Single-lead classifier, ii) 12-lead classifier, and iii) Semantic segmentation classifier. All datasets used were publicly available datasets on Physionet. The single lead and 12-lead classifiers output predicted probabilities, shown in the report as probability bins: 0-30%: Not detected; 30-45%: Cannot rule out; 45-60%: Consider; 60-75%: Possible; and 75-100%: Consistent. The study used the bins to provide a more flexible approach to decision-making by having the outputs of the system in a more nuanced manner, similar to real-world annotations that are often not binary. Interpretability results from Grad-CAM are available for the 12-lead classifier, and uncertainty awareness results from Monte Carlo Dropout are shown in the output report.

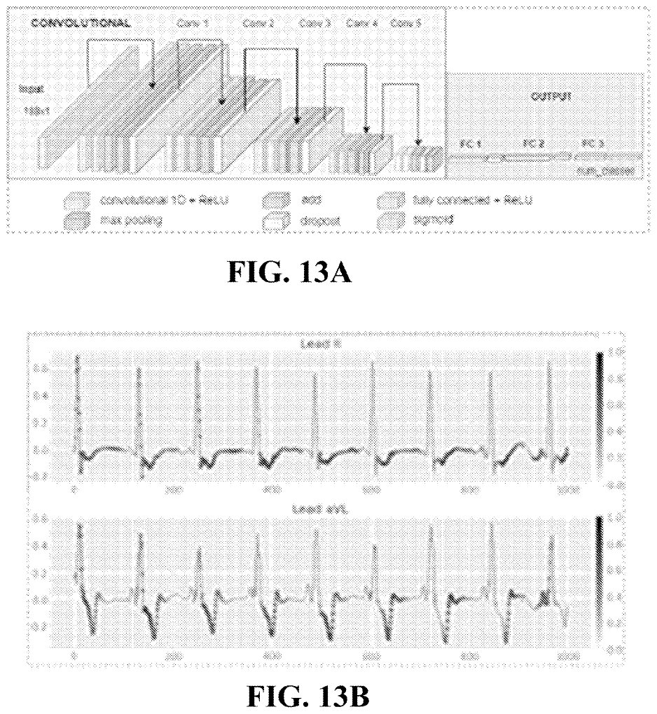

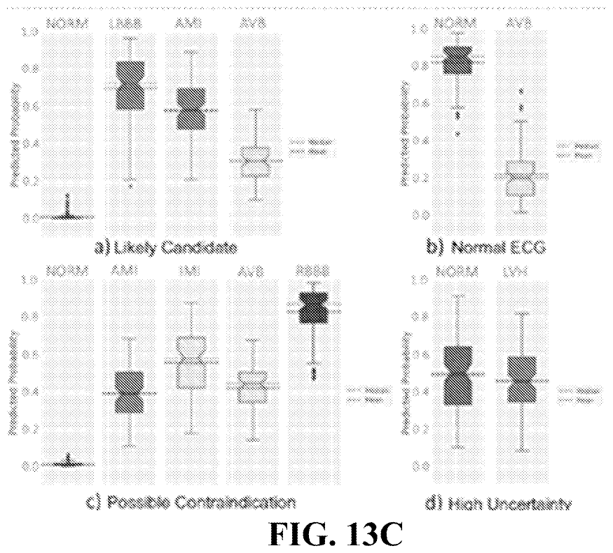

Single-lead model. Some diagnoses of interest may be correctly classified from a single lead, and high-quality datasets are available with beat-level annotation for them. The model used for the single lead classifier is based on a 1D Convolutional Neural Network with residual blocks. FIG. 13A shows a single-lead ECG classification mode. The model includes 5 layers with 1D convolutional layers, max pooling, dropout, and skip connections that are then followed by fully connected layers and sigmoid activation.

After using Keras Tuner to find the best combination of hyperparameters, a model with 64 filters, a Kernel size of 6, a MaxPool size of 4, and 48/80 units in the last two dense layers was used. The training was performed using the Adam optimizer, with a learning rate scheduled with an exponential decay rate (0.0001 initial learning rate, 0.75 rate), with callback monitoring validation loss, for 30 epochs. The loss function employed was Binary Cross-entropy.

12 lead model. The model employe the single-lead classifier, adding a sixth level of depth, with 1D-convolutional layers, implementing a group of layers for every input channel, i.e., every lead. After concatenation of the extracted features of the 12 channels, dense layers were added along with a final sigmoid activation for multilabel classification. Training was performed using the Adam optimizer, with a learning rate scheduled with an exponential decay rate (0.0001 initial learning rate, 0.75 rate), with callback monitoring validation loss for 15 epochs. The loss function employed was Binary Cross-entropy.

Semantic segmentation model. For semantic segmentation of the ECG signals, the study adapted the U-net model to process the 1D signals of each ECG lead to determine the QRS average duration in milliseconds. It comprises 5 levels of grouped max-pooling, 1D-convolutional, and dropout layers and performs upsampling with 1D-transposed convolutional layers. It receives as input a single lead waveform of 1000 samples and outputs a segmentation mask of background, P segment, QRS segment, and T segment for each of the 1000 samples from the last Softmax activation layer. The training was performed using the Adam optimizer, with a learning rate scheduled with an exponential decay rate (0.0001 initial learning rate, 0.75 rate), with callback monitoring validation loss for 85 epochs with a batch size of 64. Categorical Cross-entropy was used as the loss function.

Datasets. The study selected public ECG datasets from Physionet to evaluate the diagnoses of interest. To increase the robustness of the model each diagnosis of interest had observations from at least two datasets included in the training of the models. The datasets used included: (i) MIT-BIH Arrhythmia Database: Normal, Other beat, LBBB, RBBB, PVC, (ii) MIT-BIH Supraventricular Arrhythmia Database: Nor-mal, Other beat, PVC, (iii) PTB Diagnostic ECG Database: Normal, AMI, IMI, CHF, (iv) PTB-XL: Normal, LBBB, RBBB, LVH, RVH, AVB, RAE/RAO, AMI, IMI, (v) Lobachevsky University Electrocardiography Database (LUDB): Normal, LBBB, RBBB, LVH, RVH, AVB, RAE, PVC, AMI, QRS duration (semantic segmentation of P, QRS, T segments), and (vi) BIDMC Congestive Heart Failure Database: CHF.

The study performed training, validation, and test split sets in an approximately 0.7/0.2/0.1 split for the single-lead and semantic segmentation models and a 0.8/0.1/0.1 split for the 12-lead model. In all cases, the study stratified the split, keeping the same ratio among all classes in each subset of the data. In all cases, the study used an inter-patient paradigm, i.e., the ECG data (the heartbeats) of a patient may be used in one of the training, validation, or test sets to avoid data leakage. For two minority classes (RVH and RAE), augmentation was performed by doing a slight stretch that expands the signal in the time domain (with a random factor of 1.05 to 1.3); and scaling (by a random factor be-tween −0.875 and 1.125). The augmentations have been applied only to the 12-lead classifier.

As preprocessing, for the single lead classifier, the study detected the R-peaks and segmented the beats. For both single and 12-lead classifiers, the study used a 4th-order Butterworth high-pass filter and wavelet filtering to denoise both single and 12-lead classifiers. For the single lead and semantic segmentation models, the study applied min-max normalization.

Model Evaluation Results. The study evaluated the three models following the recommended metrics for each case. Precision, Recall, F-1 Score, and AUC per class are evaluated. Table 1 shows the obtained results for the three models. Additionally, the Intersection-Over-Union (IoU) score is calculated for the Semantic Segmentation model, taking the ratio of lengths instead of areas measuring the overlap between the predicted and ground truth regions. The obtained IoU scores from the test set with the semantic segmentation model are 0.902, 0.741, 0.867, and 0.786 for Other (Background), P, QRS, and T segments, respectively. The weighted IoU score is 0.871.

TABLE 1
Precision, Recall, F-1 and AUC results for the Single-
lead, 12-lead and Semantic Segmentation models.

| | | | | |
|---|---|---|---|---|
| Class | Precision | Recall | F1-score | AUC |

Single Lead Classifier

| | | | | |
|---|---|---|---|---|
| Normal | 0.91 | 0.99 | 0.95 | 0.994 |
| Other | 0.97 | 0.93 | 0.95 | 0.997 |
| LBBB | 1 | 1 | 1 | 1 |
| RBBB | 1 | 1 | 1 | 1 |
| PVC | 0.97 | 0.96 | 0.97 | 0.998 |
| CHF | 0.99 | 0.96 | 0.97 | 0.989 |
| MI | 0.96 | 0.96 | 0.96 | 0.997 |

12 Lead Classifier

| | | | | |
|---|---|---|---|---|
| Normal | 0.87 | 0.92 | 0.90 | 0.963 |
| LVH | 0.74 | 0.73 | 0.73 | 0.959 |
| RBBB | 0.90 | 0.82 | 0.86 | 0.988 |
| IMI | 0.75 | 0.78 | 0.77 | 0.950 |
| AMI | 0.79 | 0.82 | 0.80 | 0.969 |
| AVB | 0.52 | 0.57 | 0.55 | 0.956 |
| RVH | 0.13 | 0.83 | 0.22 | 0.945 |
| RAO/RAE | 0.43 | 0.50 | 0.46 | 0.945 |
| LBBB | 0.95 | 0.86 | 0.90 | 0.989 |

Semantic Segmentation

| | | | | |
|---|---|---|---|---|
| Other | 0.97 | 0.97 | 0.97 | 0.9918 |
| P | 0.88 | 0.90 | 0.89 | 0.9972 |
| QRS | 0.93 | 0.94 | 0.94 | 0.9990 |
| T | 0.90 | 0.90 | 0.90 | 0.9946 |

Output. The study implemented interpretability with Grad-CAM, applied to the last 1D-convolutional layer of each of the 12 heads of the model, one per input lead. FIG. 13B shows example results of the interpretability plotted over the 1D signal of the lead being examined with a heatmap.

The Monte Carlo (MC) dropout technique may yield a good approximation of the posterior probability distribution of a model. This was implemented in the system and plotted with notched box-plots. The observation was forward-passed through the model 100 times. At inference time, the system outputted a report highlighting the major, minor, and potential contraindication criteria, along with the saliency maps obtained. The physician may also inspect the boxplots of the reported criteria of interest, which also shows the estimated uncertainty of the results. FIG. 13C shows examples: one of a high candidacy result (a), a second one of a heart predicted as normal (b), a third one of a candidate with high potential contraindication (c), and the last one was an example of a high uncertainty observation (d). Specifically, FIG. 13C, subpanel A reads: Major—Consistent with LBBB, Possible AMI, QRS duration 167 ms. Minor—Cannot rule out AVB. FIG. 13C, subpanel B reads: Major—Consistent with NORMAL, QRS duration 112.5 ms (the next predicted probability is shown for com-parison, AVB with low value). FIG. 13C, subpanel C reads: Major—Cannot rule out AMI, QRS duration 128.6 ms. Minor—Possible IMI, Consider AVB. Potential Contraindications—Consistent with RBBB. FIG. 13C, subpanel D reads: Major—Consider NORMAL (high uncertainty), Consider LVH.

The study showed steps in an approach to assessing candidacy for LVAD implantation directly from physiological signals, in this case, from the ECG. A multi-model Deep Learning system was built, achieving state-of-the-art results on each model and combining the predictions into a report. The study confirmed the importance of having Interpretability and implemented a way of showing per lead and segment importance. To increase trust in the model, the study implemented uncertainty calculation, reporting graphically the predicted probability and uncertainty.

Machine Learning. Various analysis systems can be implemented using one or more artificial intelligence and machine learning operations. The term “artificial intelligence” can include any technique that enables one or more computing devices or computing systems (i.e., a machine) to mimic human intelligence. Artificial intelligence (AI) includes but is not limited to knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders and embeddings. The term “deep learning” is defined herein to be a subset of machine learning that enables a machine to automatically discover representations needed for feature detection, prediction, classification, etc., using layers of processing. Deep learning techniques include but are not limited to artificial neural networks or multilayer perceptron (MLP).

Machine learning models include supervised, semi-supervised, and unsupervised learning models. In a supervised learning model, the model learns a function that maps an input (also known as feature or features) to an output (also known as target) during training with a labeled data set (or dataset). In an unsupervised learning model, the algorithm discovers patterns among data. In a semi-supervised model, the model learns a function that maps an input (also known as a feature or features) to an output (also known as a target) during training with both labeled and unlabeled data.

Neural Networks. An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers, such as an input layer, an output layer, and optionally, one or more hidden layers with different activation functions. An ANN having hidden layers can be referred to as a deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tanh, or rectified linear unit (ReLU), and provide an output in accordance with the activation function. Additionally, each node is associated with a respective weight. ANNs are trained with a dataset to maximize or minimize an objective function. In some implementations, the objective function is a cost function, which is a measure of the ANN's performance (e.g., an error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include but are not limited to backpropagation. It should be understood that an ANN is provided only as an example machine learning model. This disclosure contemplates that the machine learning model can be any supervised learning model, semi-supervised learning model, or unsupervised learning model. Optionally, the machine learning model is a deep learning model. Machine learning models are known in the art and are therefore not described in further detail herein.

A convolutional neural network (CNN) is a type of deep neural network that has been applied, for example, to image analysis applications. Unlike traditional neural networks, each layer in a CNN has a plurality of nodes arranged in three dimensions (width, height, and depth). CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by downsampling). A fully-connected layer includes neurons, where each neuron is connected to all of the neurons in the previous layer. The layers are stacked similarly to traditional neural networks. GCNNs are CNNs that have been adapted to work on structured datasets such as graphs.

Other Supervised Learning Models. A logistic regression (LR) classifier is a supervised classification model that uses the logistic function to predict the probability of a target, which can be used for classification. LR classifiers are trained with a data set (also referred to herein as a “dataset”) to maximize or minimize an objective function, for example, a measure of the LR classifier's performance (e.g., an error such as L1 or L2 loss), during training. This disclosure contemplates that any algorithm that finds the minimum of the cost function can be used. LR classifiers are known in the art and are therefore not described in further detail herein.

A Naïve Bayes' (NB) classifier is a supervised classification model that is based on Bayes' Theorem, which assumes independence among features (i.e., the presence of one feature in a class is unrelated to the presence of any other features). NB classifiers are trained with a data set by computing the conditional probability distribution of each feature given a label and applying Bayes' Theorem to compute the conditional probability distribution of a label given an observation. NB classifiers are known in the art and are therefore not described in further detail herein.

A k-NN classifier is an unsupervised classification model that classifies new data points based on similarity measures (e.g., distance functions). The k-NN classifiers are trained with a data set (also referred to herein as a “dataset”) to maximize or minimize a measure of the k-NN classifier's performance during training. This disclosure contemplates any algorithm that finds the maximum or minimum. The k-NN classifiers are known in the art and are therefore not described in further detail herein.

In addition, the various analysis systems can be implemented encoder, transformer, and other convolutional deep neural network architecture.

### Discussion

Cardiovascular disease (CVD) is the leading cause of death in the USA [1′]. Common cardiovascular diseases include heart attack, stroke, arrhythmia, and heart valve stenosis, which all might eventually lead to heart failure (HF). It was estimated the total direct medical costs of HF would reach $53 billion by 2030 [2′]. When HF reaches the end stage, no known medicine is effective in treating it. Cardiac transplant surgery is the gold standard treatment for end-stage heart failure but is severely limited by the availability of donor hearts. Moreover, there are no readily available methods to increase the pool of donor hearts. As a promising alternative treatment, left ventricular assist device (LVAD) implants have been steadily rising from 2010 to 2021, amounting to a total of 25,551 in the period in the US, and are expected to reach 7,000 per year by 2030 [3′], [4′]. The tiny implantable pump helps a failing heart to sustain blood circulation to the body, but the current generation of LVADs are invasive, which is primarily caused by the percutaneous driveline and frequent emergency pump exchange surgeries to treat thrombosis-related pump failure [5′], [6′]. Common complications, including infection, thrombosis, stroke, and bleeding, greatly reduce the quality of life for LVAD patients. Significant research efforts need to be carried out to make LVAD therapy less invasive, more effective, and more accessible to the public and military. The overarching challenges facing the development of less invasive LVADs are: 1) Blood damage caused by the mechanical force and associated adverse effects, including bleeding and thrombosis, requiring hospitalization and emergency surgery. The blood damage is associated with the supraphysiological shear stress [3′], [7′] in the device, and the high mechanical force destroys blood cells, leading to hemolysis, and activates platelets, causing thrombosis; 2) Pump thrombosis, which often leads to emergency pump exchanges and surgeries. One of the root causes of LVAD failures is the presence of blood stagnation points, which trigger blood protein adsorption followed by a series of chain reactions, including thrombosis, ultimately leading to sepsis and death; 3) The percutaneous driveline, which causes infection and usually leads to rehospitalization and surgeries. Besides, driveline greatly reduces the mobility of patients, thereby declining their quality of life.

To address the plethora of problems faced by the current generation of LVADs, more than a single innovation is required to significantly improve clinical outcomes. The exemplary device incorporates innovations in hemodynamics, surface science, electronics, and hemocompatibility to accomplish the collaborative goal of developing a less invasive, highly durable, and effective LVAD to enhance the quality of life for HF patients, including (i) improving the hemocompatibility and eliminating the thrombosis problem at the inlet cannula by adopting innovative design changes, including flexible blades/casing and a stented inlet, (ii) using slippery hydrophilic (SLIC) coatings with unprecedented antithrombotic properties, both in quasi-static and dynamic conditions on LVAD surfaces, addressing the issue of thrombosis within the device, and (iii) using the external power source and transmitter structure to wireless power the implanted pump and the communication link for the centrifugal pump to provide feedback to the external power system.

The elimination of percutaneous driveline and ultra-low risk of thrombosis-related pump-exchange surgeries make the long-term support by an LVAD much less invasive. It also provides a much-improved quality of life to patients supported by LVADs. Furthermore, the flexible rotor and casing design opens the door for the future development of minimally invasive LVAD that can be implanted via a catheter. The innovation will have a profound impact on how we treat heart failure patients and benefit the US public.

Literature Discussion. The exemplary device can use fabric to promote endothelialization to reduce the risk of thrombosis. In contrast, U.S. Pat. No. 9,114,034 B2 appears to disclose coating the stent with antithrombotic agents to reduce the possibility of thrombosis. US 2016/0303287 appears to disclose coating the stent with an antithrombotic agent to reduce the thrombosis risk of a stented heart valve.

The exemplary device can be used for reducing flow stasis and thrombosis near the LVAD inlet. In contrast, WO 2020/127616A1 discloses a prosthetic heart valve having a stent structure with a conical-convex inflow region and a linear cylindrical outflow region.

The exemplary device may employ a flexible rotor to reduce the blood damage and the outcome of LVAD patients. In contrast, U.S. Pat. No. 8,449,443B2 discloses a pump with a flexible rotor to support the Fontan circulation. The propeller appears to be flexible to facilitate implantation intravascularly with crimping.

### CONCLUSION

It must also be noted that, as used in the specification and the appended claims, the singular forms “a,” “an,” and “the” include plural referents unless the context clearly dictates otherwise. Ranges may be expressed herein as from “about” or “5 approximately” one particular value and/or to “about” or “approximately” another particular value. When such a range is expressed, other exemplary embodiments include one particular value and/or the other particular value.

By “comprising” or “containing” or “including,” is meant that at least the name compound, element, particle, or method step is present in the composition or article or method but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The following patents, applications and publications as listed below and throughout this document are hereby incorporated by reference in their entirety herein.

### REFERENCE LIST #1

[1] US 2016/0303287
[2] U.S. Pat. No. 10,442,166 B2
[3] U.S. Pat. No. 4,906,237
[4] U.S. Pat. No. 5,662,960
[5] U.S. Pat. No. 11,285,312B2
[6] U.S. Pat. No. 9,814,611 B2
[7] US 2011/0276123
[8] U.S. Pat. No. 10,434,235B2
[9] U.S. Pat. No. 11,065,462B2
[10] U.S. Pat. No. 7,520,850B2
[11] Tsao C. W. et al. Circulation, vol. 145, no. 8, pp. e153-e639, 2022.
[12] Sahni A. et al. Annals of Biomedical Engineering, 2023
[13] Guglin M. The VAD Journal, vol. 8, no. 1, p. 1, 2022.

### REFERENCE LIST #2

[1′] MacPhee D W and Beyene A. Performance analysis of a small wind turbine equipped with flexible blades. Renewable Energy. 2019; 132:497-508.
[2′] Heron M. Deaths: Leading Causes for 2018. Natl Vital Stat Rep. 2021; 2021 May: 1-115.
[3′] Heidenreich P A, Albert N M, Allen L A, Bluemke D A, Butler J, Fonarow G C, Ikonomidis J S, Khavjou O, Konstam M A, Maddox T M, Nichol G, Pham M, Pina I L, Trogdon J G, American Heart Association Advocacy Coordinating C, Council on Arteriosclerosis T, Vascular B, Council on Cardiovascular R, Intervention, Council on Clinical C, Council on E, Prevention and Stroke C. Forecasting the impact of heart failure in the United States: a policy statement from the American Heart Association. Circ Heart Fail. 2013; 6:606-19.
[4′] Goldstein D J, Meyns B, Xie R, Cowger J, Pettit S, Nakatani T, Netuka I, Shaw S, Yanase M and Kirklin J K. Third annual report from the ISHLT Mechanically Assisted Circulatory Support Registry: a comparison of centrifugal and axial continuous-flow left ventricular assist devices. The Journal of Heart and Lung Transplantation. 2019; 38:352-363.
[5′] Gustafsson F and Rogers J G. Left ventricular assist device therapy in advanced heart failure: patient selection and outcomes. European journal of heart failure. 2017; 19:595-602.
[6′] Kirklin J K, Naftel D C, Pagani F D, Kormos R L, Myers S, Acker M A, Rogers J, Slaughter M S and Stevenson L W. Pump thrombosis in the Thoratec HeartMate II device: An update analysis of the INTERMACS Registry. J Heart Lung Transplant. 2015; 34:1515-26.
[7′] Koval C E, Thuita L, Moazami N and Blackstone E. Evolution and impact of drive-line infection in a large cohort of continuous-flow ventricular assist device recipients. J Heart Lung Transplant. 2014; 33:1164-72.
[8′] Eckman P M and John R. Bleeding and thrombosis in patients with continuous-flow ventricular assist devices. Circulation. 2012; 125:3038-47.
[9′] Welle G A, El-Sabawi B, Thaden J J, Greason K L, Klarich K W, Nkomo V T, Alkhouli M A, Guerrero M E, Crestanello J A, Holmes D R, Jr., Rihal C S and Eleid M F. Effect of a fourth-generation transcatheter valve enhanced skirt on paravalvular leak. Catheter Cardiovasc Interv. 2021; 97:895-902.
[10.′] Mahtta D and Jneid H. Paravalvular leak after TAVR: remarkable improvement but not the time to shift focus. Catheter Cardiovasc Interv. 2021; 97:903-904.
[11′] Otsuka F, Finn A V, Yazdani S K, Nakano M, Kolodgie F D and Virmani R. The importance of the endothelium in atherothrombosis and coronary stenting. Nat Rev Cardiol. 2012; 9:439-53.
[12′] Stoeckel D, Pelton A and Duerig T. Self-expanding nitinol stents: material and design considerations. Eur Radiol. 2004; 14:292-301.
[13′] Marchand C, Heim F, Durand B and Chafke N. Nitinol Stent for Percutaneous Heart Valve Implantation: Material Shape Setting. Materials and Manufacturing Processes. 2011; 26:181-187.
[14′] Taylor G, Wang Z, Vardaki E and Gursul I. Lift Enhancement over Flexible Nonslender Delta Wings. AIAA Journal. 2007; 45:2979-2993.
[15′] Gursul I. Recent developments in delta wing aerodynamics. The Aeronautical Journal. 2004; 108:437-452.
[16′] Katz J and Weihs D. Hydrodynamic propulsion by large amplitude oscillation of an airfoil with chordwise flexibility. Journal of Fluid Mechanics. 2006; 88:485-497.
[17′] Ifju P, Jenkins D, Ettinger S, Lian Y and Shyy W. Flexible-wing-based micro air vehicles. Paper presented at: 40th AIAA aerospace sciences meeting & exhibit; 2002; Reno, NV.
[18′] Açιkel HH and Serdar Genç M. Control of laminar separation bubble over wind turbine airfoil using partial flexibility on suction surface. Energy. 2018; 165:176-190.
[19′] Hsu P-L, McIntyre M, Kuetting M, Parker J, Egger C, Autschbach R, Schmitz-Rode T and Steinseifer U. Review of Recent Patents on Foldable Ventricular Assist Devices. Recent Patents on Biomedical Engineering. 2012; 5:208-222.
[20′] Hsu P L, Wang Y, Amaral F, Parker J, Schmitz-Rode T, Autschbach R and Steinseifer U. Design method of a foldable ventricular assist device for minimally invasive implantation. Artif Organs. 2014; 38:298-308.
[21′] Schmitz-Rode T, Graf J, Pfeffer J G, Buss F, Brucker C and Gunther R W. An expandable percutaneous catheter pump for left ventricular support: proof of concept. J Am Coll Cardiol. 2005; 45:1856-61.
[22′] Throckmorton A L, Ballman K K, Myers C D, Frankel S H, Brown J W and Rodefeld M D. Performance of a 3-bladed propeller pump to provide cavopulmonary assist in the failing Fontan circulation. Ann Thorac Surg. 2008; 86:1343-7.
[23′] Throckmorton A L, Kapadia J Y, Carr J P, Powell C M, Tate R D and Traynham D V. Flexible Impeller Blades in an Axial Flow Pump for Intravascular Cavopulmonary Assistance of the Fontan Physiology. Cardiovascular Engineering and Technology. 2010; 1:244-255.
[24′]. Gülich J. Centrifugal Pumps. 2 ed. Berlin Heidelberg: Springer-Verlag 2010.
[25′] Brzoska J, Azouz I B and Rondelez F. Silanization of solid substrates: a step toward reproducibility. Langmuir. 1994; 10:4367-4373.
[26′] Fadeev A Y and McCarthy T J. Trialkylsilane monolayers covalently attached to silicon surfaces: wettability studies indicating that molecular topography contributes to contact angle hysteresis. Langmuir. 1999; 15:3759-3766.
[27′] Brzoska J, Shahidzadeh N and Rondelez F. Evidence of a transition temperature for the optimum deposition of grafted monolayer coatings. Nature. 1992; 360:719-721.
[28′] Fadeev A Y and Mccarthy T J. Self-assembly is not the only reaction possible between alkyltrichlorosilanes and surfaces: monomolecular and oligomeric covalently attached layers of dichloro- and trichloroalkylsilanes on silicon. Langmuir. 2000; 16:7268-7274.
[29′] Onclin S, Ravoo B J and Reinhoudt D N. Engineering silicon oxide surfaces using Self-Assembled monolayers. Angewandte Chemie International Edition. 2005; 44:6282-6304.
[30′] Wasserman S R, Tao Y T and Whitesides G M. Structure and reactivity of alkylsiloxane monolayers formed by reaction of alkyltrichlorosilanes on silicon substrates. Langmuir. 1989; 5:1074-1087.
[31′] Britt D W and Hlady V. An AFM Study of the Effects of Silanization Temperature, Hydration, and Annealing on the Nucleation and Aggregation of Condensed OTS Domains on Mica. J Colloid Interface Sci. 1996; 178:775-784.
[32′] Kallury K M, Macdonald P M and Thompson M. Effect of surface water and base catalysis on the silanization of silica by (aminopropyl) alkoxysilanes studied by X-ray photoelectron spectroscopy and 13C cross-polarization/magic angle spinning nuclear magnetic resonance. Langmuir. 1994; 10:492-499.
[33′] Zhuravlev L. Concentration of hydroxyl groups on the surface of amorphous silicas. Langmuir. 1987; 3:316-318.
[34′] Rye R, Nelson G and Dugger M. Mechanistic aspects of alkylchlorosilane coupling reactions. Langmuir. 1997; 13:2965-2972.
[35′] Liston E, Martinu L and Wertheimer M. Plasma surface modification of polymers for improved adhesion: a critical review. Journal of adhesion science and technology. 1993; 7:1091-1127.
[36] Strobel M, Lyons C S and Mittal K L. Plasma surface modification of polymers: relevance to adhesion. 1994.0
[37′] Cvelbar U, Pejovnik S, Mozetie M and Zalar A. Increased surface roughness by oxygen plasma treatment of graphite/polymer composite. Applied surface science. 2003; 210:255-261.
[38′] Levchenko I, Xu S, Baranov O, Bazaka O, Ivanova EP and Bazaka K. Plasma and polymers: recent progress and trends. Molecules. 2021; 26:4091.
[39′] Vesel A and Mozetic M. Surface modification and ageing of PMMA polymer by oxygen plasma treatment. Vacuum. 2012; 86:634-637.
[40′] Yang P and Yang W. Hydroxylation of organic polymer surface: method and application. ACS Applied Materials & Interfaces. 2014; 6:3759-3770.
[41′] Duan S, Liu X, Wang Y, Meng Y, Alsaedi A, Hayat T and Li J. Plasma surface modification of materials and their entrapment of water contaminant: A review. Plasma Processes and Polymers. 2017; 14:1600218.
[42′] Cheng C, Liye Z and Zhan R-J. Surface modification of polymer fibre by the new atmospheric pressure cold plasma jet. Surface and Coatings Technology. 2006; 200:6659-6665.
[43′] Cha H, Vahabi H, Wu A, Chavan S, Kim M-K, Sett S, Bosch S A, Wang W, Kota A K and Miljkovic N. Dropwise condensation on solid hydrophilic surfaces. Science Advances. 2020; 6:eaax0746.
[44′] Movafaghi S, Cackovic M D, Wang W, Vahabi H, Pendurthi A, Henry C S and Kota A K. Superomniphobic Papers for On-Paper pH Sensors. Adv Mater Interfaces. 2019; 6:1900232.
[45′] Vahabi H, Wang W, Movafaghi S and Kota A K. Free-Standing, Flexible, Superomniphobic Films. ACS Applied Materials & Interfaces. 2016; 8:21962-21967.
[46′] Vahabi H, Wang W, Davies S, Mabry J M and Kota A K. Coalescence-Induced Self-Propulsion of Droplets on Superomniphobic Surfaces. ACS Applied Materials & Interfaces. 2017; 9:29328-29336.
[47′] Wang W, Du X, Vahabi H, Zhao S, Yin Y, Kota A K and Tong T. Trade-off in membrane distillation with monolithic omniphobic membranes. Nature Communications. 2019; 10:3220.
[48′] Wang W, Lockwood K, Boyd L M, Davidson M D, Movafaghi S, Vahabi H, Khetani S R and Kota A K. Superhydrophobic Coatings with Edible Materials. ACS Applied Materials & Interfaces. 2016; 8:18664-18668.
[49] Wang W, Salazar J, Vahabi H, Joshi-Imre A, Voit W E and Kota A K. Metamorphic Superomniphobic Surfaces. Advanced Materials. 2017; 29:1700295.
[50′] Wang W, Vahabi H, Movafaghi S and Kota A K. Superomniphobic surfaces with improved mechanical durability: Synergy of hierarchical texture and mechanical interlocking. Adv Mater Interfaces. 2019; 6:1900538.
[51′] Wang W, Du X, Vahabi H, Zhao S, Yin Y, Kota A K and Tong T. Trade-off in membrane distillation with monolithic omniphobic membranes. Nature communications. 2019; 10:1-9.
[52′] Vallabhuneni S, Movafaghi S, Wang W and Kota A K. Superhydrophobic coatings for improved performance of electrical insulators. Macromolecular Materials and Engineering. 2018; 303:1800313.
[53′] Bartlet K, Movafaghi S, Dasi L P, Kota A K and Popat K C. Antibacterial activity on superhydrophobic titania nanotube arrays. Colloids Surf B Biointerfaces. 2018; 166:179-186.
[54′ Kota A K, Kwon G and Tuteja A. The design and applications of superomniphobic surfaces. NPG Asia Materials. 2014; 6:e109.
[55′] Kota A K, Mabry J M and Tuteja A. Superoleophobic surfaces: design criteria and recent studies. Surface Innovations. 2013; 1:71-83.
[56′] Tadmor R, Das R, Gulec S, Liu J, E. N'guessan H, Shah M, S. Wasnik P and Yadav SB. Solid-Liquid Work of Adhesion. Langmuir. 2017; 33:3594-3600.
[57′] Vahabi H, Wang W, Popat K C, Kwon G, Holland T B and Kota A K. Metallic superhydrophobic surfaces via thermal sensitization. Applied Physics Letters. 2017; 110:251602.
[58′] Howarter J A and Youngblood J P. Optimization of silica silanization by 3-aminopropyltriethoxysilane. Langmuir. 2006; 22:11142-11147.
[59′] Ferrari G, De Lazzari C, Kozarski M, Clemente F, Gorczynska K, Mimmo R, Monnanni E, Tosti G and Guaragno M. A hybrid mock circulatory system: testing a prototype under physiologic and pathological conditions. ASAIO J. 2002; 48:487-94.
[60′] Yaxin W, Smith P A, De-Sciscio P, Sampaio L C, Cohn W E, Liping X and McMahon R A. Replication of pressure-volume loop with controllable ESPVR and EDPVR curves on a personalized mock circulatory loop based on elastance function. Annu Int Conf IEEE Eng Med Biol Soc. 2017; 2017:1282-1286.
[61′] Cuenca-Navalon E, Finocchiaro T, Laumen M, Fritschi A, Schmitz-Rode T and Steinseifer U. Design and evaluation of a hybrid mock circulatory loop for total artificial heart testing. Int J Artif Organs. 2014; 37:71-80.
[62′] Pauls J P, Stevens M C, Bartnikowski N, Fraser J F, Gregory S D and Tansley G. Evaluation of Physiological Control Systems for Rotary Left Ventricular Assist Devices: An In-Vitro Study. Ann Biomed Eng. 2016; 44:2377-2387.
[63′] Wang Y, Smith P A, Timms D L, Hsu P L and McMahon R A. In Vitro Evaluation of the Dual-Diffuser Design for a Reversible Rotary Intra-Aortic Ventricular Assist Device. Artif Organs. 2016; 40:884-93.

1. An implantable left ventricular assist device (LVAD) comprising:
a pump housing forming a volume therein, the pump housing having an inlet and an outlet;
a rotor disposed within the pump housing to rotate in the volume to pump blood;
a motor coupled to the rotor to drive rotation of the rotor; and
an inlet member coupled to the inlet of the housing, the inlet member having (i) an external structure configured to conform and contact an outer surface wall at a base of a left ventricle and (ii) an internal expandable body configured to move between a stowed configuration and a deployed configuration to be placed and extending into the left ventricle, wherein the internal expandable body is defined by (i) a first section having a first circumference sized to couple to the inlet of the pump and (ii) a second section having a second circumference sized, wherein in the deployed configuration, to substantially contact an inner surface wall of the left ventricle to eliminate flow stasis in the left ventricle.

2. The device of claim 1 wherein the rotor comprises:
a rotor body; and
a plurality of curved flexible blades extending therefrom, wherein the plurality of curved flexible blades are flexibly shaped and formed of a flexible material, to reduce shear stress on components of the blood.

3. The device of claim 1 wherein the rotor comprises an antithrombotic coating comprising a hydrophilic and slippery polymer.

4. The device of claim 1, further comprising:
a drive system configured to drive the rotor to rotate in the volume of the pump housing; and
an implanted control unit that electrically couples to the drive system to control the drive system and rotation of the rotor, wherein the implanted control unit includes:
one or more energy storage devices;
a charging circuit; and
a wireless power transfer circuit comprises an antenna coil and electronics to control inductive charging operation with an external RF source.

5. The device of claim 2, wherein the motor comprises a maglev drive subassembly comprising:
one or more permanent magnets located in the rotor body;
a stator disposed at a position in the pump housing proximal to the one or more permanent magnets; and
one or more magnetic bearings disposed in one or more positions in the pump housing proximal to the plurality of curved flexible blades of the rotor.

6. The device of claim 1, wherein the internal expandable body of the inlet member comprises a flexible mesh surrounded by or embedded in a fabric, wherein the flexible mesh is configured as self-expanding or balloon-expanding.

7. The device of claim 6, wherein the internal expandable body of the inlet member is antithrombotic and anti-restenotic drug eluting.

8. The device of claim 6, after deployment, the internal expandable body is defined by (i) a first section having a first circumference sized to couple to the inlet of the pump and (ii) a second section having a second circumference sized to continuously contact and conform to the inner surface wall of the left ventricle, to conform to a shape of the left ventricle, and wherein the internal expandable body is configured to deform with ventricular motions.

9. (canceled)

10. The device of claim 6, wherein the fabric comprises a resorbable material to promote endothelialization.

11. The device of any claim 1, wherein the internal expandable body comprises a resorbable material to promote endothelialization.

12. The device of claim 10, wherein the fabric includes polyester, polytetrafluoroethylene, or a combination thereof.

13. The device of claim 4, wherein the internal expandable body, including flexible mesh and fabric, is patient-specifically designed to be sized and shaped to a configuration to match a scan of the left ventricle of a patient.

14. The device of claim 1, wherein the pump housing and/or the inlet member comprises:
a quick disconnect connector to releasably and attachably connect the pump housing to the inlet member.

15. The device of claim 2, wherein the pump housing has (i) a flexible external structure and (ii) an internal deformable member disposed within a portion of a surface defining the volume of the pump housing, the internal deformable member comprising a flexible material.

16. (canceled)

17. (canceled)

18. The device of claim 15, wherein the portion of the surface defining the volume of the pump housing having the flexible material corresponds to regions of contact with the plurality of curved flexible blades during rotation of the plurality of curved flexible blades.

19. The device of claim 15, wherein the portion of the surface defining the volume of the pump housing having the flexible material corresponds to regions of contact with the plurality of curved flexible blades during (i) rotation of the plurality of curved flexible blades and (ii) off-axis movement of the plurality of curved flexible blades mounted configured to move off-axis via a magnetic bearing and electromagnetic driving subsystem.

20. (canceled)

21. The device of claim 15, wherein internal surfaces of the pump housing, including the internal deformable member, comprises an antithrombotic coating comprising a hydrophilic and slippery polymer.

22. The device of claim 21, wherein the antithrombotic coating of interface surfaces of the pump housing and/or the antithrombotic coating of the rotor is optimized for a maximum antithrombotic response via systematic tailoring of hydroxylation parameters, molecular architecture, and synthesis reaction coordinates.

23. The device of claim 22, wherein the antithrombotic coating of the interface surfaces of the pump housing and/or the antithrombotic coating of the rotor is formed via liquid-phase silanization of a flexible material of the interface surfaces of the pump housing and/or the antithrombotic coating of the rotor to form a silane.

24. The device of claim 23, wherein the silane is tuned (i) to obtain high grafting densities to the flexible material of the interface surfaces of the pump housing and/or the antithrombotic coating of the rotor and (ii) to ensure an antithrombotic nature.

25. (canceled)

26. The device of claim 13, further comprising:
an implantable subcutaneous coil,
wherein the wireless power transfer circuit of the implanted control unit is operatively coupled via a drive line conductor to the implantable subcutaneous coil, the implantable subcutaneous coil being configured to operate as a pair of coupled coils with an external coil configured to be affixed (i) to a skin region of the patient and (ii) in proximity to the implantable subcutaneous coil.

27. The device of claim 4, wherein the one or more energy storage devices include (i) one or more rechargeable batteries and (ii) at least one of a supercapacitor and a hybrid supercapacitor, the one or more rechargeable batteries and the at least one of the supercapacitor and the hybrid supercapacitor having a combined energy storage of at least 20 minutes.

28. The device of claim 26, wherein the implanted control unit comprises (i) a processor and (ii) a memory having instructions stored thereon,
wherein execution of the instructions by the processor causes the processor to execute a dynamic charging algorithm.

29. The device of claim 26, wherein the implanted control unit comprises a wireless communication interface configured to operatively connect with a remote controller.

30. The device of claim 29, wherein the remote controller has (i) a processor and (ii) a memory having instructions stored thereon,
wherein execution of the instructions by the processor causes the processor to:
execute a dynamic charging algorithm comprising a trained machine learning model or a model derived therefrom; and
transmit an output of the dynamic charging algorithm to the implanted control unit to adjust control operation thereat.

31. The device of claim 29, wherein the implanted control unit is configured to execute a dynamic charging algorithm configured to reduce LVAD speed during time of less patient activity.

32. The device of claim 30, wherein the dynamic charging algorithm includes a dynamic motor control loop that reduce LVAD speed during time of less patient activity, the dynamic motor control loop having an output to drive operation of the motor.

33. (canceled)

34. (canceled)

35. (canceled)

36. The device of claim 1 further comprising:
a drive system with magnetic levitation that includes a magnetically levitated ventricular assist device (VAD), comprising: an impeller attached to rotor permanent magnets; a stator embedded within a VAD housing adjacent to an inlet cannula; an active magnetic levitated bearing located at a bottom of the VAD housing with an eddy current sensor configured to monitor a gap between a impeller tip and a maglev.

37. The device of claim 1, wherein the implantable left ventricular assist device was selected to be implanted in a patient identified via a trained ML algorithm employed to evaluate candidate assessment, wherein the trained ML algorithm is configured to estimate a likelihood of a presence of a candidate criteria for an LVAD implant.

38. (canceled)

39. (canceled)
